(19) United States
(12) Patent Application Publication
(10) Pub. No.: US 2021/0121493 A1
(43) Pub. Date: Apr. 29, 2021

(54) METHODS FOR TREATING LIVER DISEASES

(71) Applicant: Cedars-Sinai Medical Center, Los Angeles, CA (US)

(72) Inventors: Mazen NOUREDDIN, Beverly Hills, CA (US); Shelly LU, Los Angeles, CA (US)

(73) Assignee: Cedars-Sinai Medical Center, Los Angeles, CA (US)

(21) Appl. No.: 16/634,042

(22) PCT Filed: Jul. 24, 2018
(86) PCT No.: PCT/US2018/043508

§ 371 Date: Jan. 24, 2020

Related U.S. Application Data
(60) Provisional application No. 62/536,649, filed on Jul. 25, 2017.

(51) Int. Cl. A61K 31/7076; A61K 31/575; A61K 31/42; A61K 31/4439; A61K 31/416; A61K 31/46; A61K 31/496; A61K 31/454; A61K 31/55; A61K 31/343; A61K 31/196; A61K 31/355; A61K 31/515; A61K 31/07; A61K 31/593; A61K 31/122; A61K 31/485; A61K 31/36; A61K 31/519; A61K 36/74; A61K 45/06; A61P 1/16; A61P 35/00

(52) U.S. Cl. CPC A61K 31/7076; A61P 35/00; A61K 31/42; A61K 31/4439; A61K 31/416; A61K 31/46; A61K 31/496; A61K 31/454; A61K 31/55; A61K 31/343; A61K 31/196; A61K 31/355; A61K 31/515; A61K 31/07; A61K 31/593; A61K 31/122; A61K 31/485; A61K 31/36; A61K 31/519; A61K 36/74; A61K 45/06; A61P 1/16; A61K 31/575

(57) ABSTRACT

Provided herein are method for treating liver diseases. The methods include administering to the subject a therapeutically effective amount of at least one FXR agonist and a therapeutically effective amount of SAMe. In various embodiments, the at least one FXR agonist and SAMe are administered sequentially or simultaneously.

### CROSS-REFERENCE TO RELATED APPLICATION

This application is a National Phase of International Application No. PCT/US2018/043508, filed Jul. 24, 2018, which designated the U.S. and that International Application was published under PCT Article 21(2) in English. Both applications include a claim of priority under 35 U.S.C. § 119(e) to U.S. Provisional Patent Application No. 62/536,649, filed Jul. 25, 2017, the entirety of which is hereby incorporated by reference.

### STATEMENT REGARDING FEDERALLY SPONSORED RESEARCH OR DEVELOPMENT

This invention was made with government support under Grant No. DK092407 awarded by the National Institutes of Health. The government has certain rights in the invention.

### TECHNICAL FIELD

Various embodiments of the invention relate to methods for treating liver diseases using a combination of at least one FXR agonist, and S-adenosylmethionine (SAMe).

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Primary biliary cholangitis (PBC) also known as primary biliary “cirrhosis”, is a rare autoimmune liver disease resulting in buildup of bile in the liver and thus contributing to inflammation and scarring (liver fibrosis). Eventually this can lead to cirrhosis and its associated complication, as scar tissue replaces healthy liver tissue and liver function becomes increasingly impaired.

Obeticholic acid (OCA), an FXR agonist, was recently approved for treatment of PBC. The inventors have shown that OCA induces expression of the oncogene MAFG and can enhance liver cancer growth. OCA also causes pruritus. There is a need for therapies that inhibit the side-effects of FXR agonists (e.g., OCA) while retaining therapeutic benefits of FXR agonists (e.g., OCA). In various embodiments, the present invention addresses that need.

### SUMMARY OF THE INVENTION

The following embodiments and aspects thereof are described and illustrated in conjunction with systems, compositions and methods which are meant to be exemplary and illustrative, not limiting in scope.

In various embodiments, the present invention provides, a method for treating liver disease in a subject in need thereof, comprising: administering to the subject, a therapeutically effective amount of at least one FXR agonist and a therapeutically effective amount of SAMe.

In some embodiments, the liver disease is selected from the group consisting of nonalcoholic fatty liver disease (NAFLD), nonalcoholic steatohepatitis (NASH), cholestatic liver disease, alcoholic liver disease, liver fibrosis, primary biliary cholangitis, pruritus, chronic hepatitis B, primary sclerosing cholangitis, and combinations thereof. In some embodiments, the at least one FXR agonist and SAMe are administered orally. In some embodiments, the at least one FXR agonist is selected from the group consisting of obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof. In some embodiments, the at least one FXR agonist is selected from the group consisting of obeticholic acid (OCA), cholic acid, tropifexor, cafestol, fexaramine, GW4064, and combinations thereof. In some embodiments, the at least one FXR agonist is selected from the group consisting of obeticholic acid (OCA), tropifexor, GW4064, and combinations thereof. In some embodiments, the subject is human. In some embodiments, the therapeutically effective amount of the at least one FXR agonist is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day. In some embodiments, the therapeutically effective amount of SAMe is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day, about 1 gm per day to about 2 gm per day. In some embodiments, SAMe is administered to the subject at a dose of about 400 mg three, four or five times per day or at a dose of 500 mg three or four times per day. In some embodiments, the at least one FXR agonist is administered to the subject at a dose of about 5 mg to 10 mg daily. In some embodiments, the SAMe is administered at about the same, 1-fold, 5-fold, or 10-fold higher concentrations than the at least one FXR agonist. In some embodiments, the at least one FXR agonist and SAMe are administered sequentially. In some embodiments, the at least one FXR agonist and SAMe are administered simultaneously. In some embodiments, SAMe is administered before and after administration of at least one FXR agonist. In some embodiments, the at least one FXR agonist and SAMe are administered before, during and/or after the subject develops the liver disease. In some embodiments, the at least one FXR agonist and SAMe are administrated to the subject 1-3 times per day or 1-7 times per week. In some embodiments, the at least one FXR agonist and SAMe are administered to the subject for 1-5 days, 1-5 weeks, 1-5 months, 1-5 years, 5-10 years or longer. In some embodiments, the method further comprises administering an existing therapy for liver disease to the subject. In some embodiments, the existing therapy is selected from the group consisting of treatment with vitamin E, pioglitazone and/or life style changes including diet, exercise and weight loss, ursodeoxycholic acid, phenobarbital, cholestyramine, life style changes including diets rich in medium-chain triglycerides, long-chain triglycerides and/or treatment with oral absorbable, fat-soluble vitamin formulation A, D, E, and K supplementation, abstinence from alcohol, cessation of smoking, weight loss and/or treatment with steroids, Naltrexone, Acamprosate, Disulfiram, Topiramate and/or baclofen, eliminating hepatitis B virus or hepatitis C virus in chronic viral hepatitis, abstaining from alcohol, removing heavy metals such as iron in hemochromatosis or copper in Wilson disease, decompressing bile ducts in biliary obstruction and/or treatment with corticosteroids, penicillamine and/or colchicine, Ursodeoxycholic acid (UDCA), liver transplant, treatment with immunosuppressant drugs including methotrexate and/or colchicine and/or treatment with fenofibrate and/or bezafibrate, coffee, and combinations thereof. In some embodiments, the method further comprises treating and/or inhibiting and/or reducing a side-effect in the subject. In some embodiments, the side-effect is associated with or results from the FXR agonist or treatment with the FXR agonist. In some embodiments, the side-effect is selected from the group consisting of liver cancer, enhanced liver cancer growth, pruritus, and combinations thereof.

In various embodiments, the present invention provides a method for assessing the efficacy of the treatment for treating liver disease in a subject provided herein, comprising comparing the severity of the side-effect in the subject to the severity of the side-effect in a control subject, wherein a decrease in the severity of the side-effect in the subject relative to the control subject is indicative of the efficacy of the treatment.

In various embodiments, the present invention provides a method for assessing the efficacy of the treatment for treating liver disease in a subject provided herein, comprising comparing the liver disease and the side-effect in the subject to the liver disease and the side-effect in a control subject, wherein a decrease in the liver disease and the side-effect in the subject relative to the control subject is indicative of the efficacy of the treatment.

In various embodiments, the present invention provides a method for treating, reducing and/or inhibiting a side-effect associated with therapeutic use of at least one FXR agonist in a subject, comprising: administering to the subject, a therapeutically effective amount of SAMe. In some embodiments, the method further comprises administering a therapeutically effective amount of at least one FXR agonist. In some embodiments, the at least one FXR agonist and SAMe are administered sequentially or simultaneously.

In various embodiments, the present invention provides a pharmaceutical composition, comprising at least one FXR agonist, and SAMe. In some embodiments, the pharmaceutical composition further comprises at least one pharmaceutically acceptable carrier. In some embodiments, the pharmaceutical composition further comprises at least one pharmaceutically acceptable excipient. In some embodiments, the at least one FXR agonist is selected from the group consisting of obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof. In some embodiments, the at least one FXR agonist is selected from the group consisting of obeticholic acid (OCA), cholic acid, tropifexor, cafestol, fexaramine, GW4064, and combinations thereof. In some embodiments, the at least one FXR agonist is selected from the group consisting of obeticholic acid (OCA), tropifexor, GW4064, and combinations thereof.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts in accordance with various embodiments of the invention, that tumor volume in mice treated with OCA is three fold larger than the tumor volume in mice treated with SAMe.

FIG. 2 depicts in accordance with various embodiments of the invention, SAMe prevents liver cancer cell growth caused by OCA.

FIG. 3 depicts in accordance with various embodiments of the invention, SAMe prevents liver cancer cell growth caused by GW (GW4064) or Tropi (Tropifexor).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Abbreviations: Obeticholic acid (OCA); Farnesoid X Receptor (FXR); S-adenosylmethionine (SAMe); Primary biliary cholangitis (PBC); nonalcoholic steatohepatitis (NASH); nonalcoholic fatty liver disease (NAFLD)

“Optional” or “optionally” means that the subsequently described circumstance may or may not occur, so that the description includes instances where the circumstance occurs and instances where it does not.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; proteins, polypeptides, or peptides in solution or bound to a substrate; a cell; a tissue; a tissue print; a fingerprint, skin or hair; fragments and derivatives thereof. Non-limiting examples of samples or biological samples include cheek swab; mucus; whole blood, blood, serum; plasma; blood products, derivatives of blood products, urine; saliva; semen; lymph; fecal extract; sputum; other body fluid or biofluid; cell sample; tissue sample, tissue extract; tissue biopsy, biopsy specimen, biopsy sample, etc. The term also includes a mixture of the above-mentioned samples or biological samples. The term “sample” also includes untreated or pretreated (or pre-processed) biological samples. In some embodiments, a sample or biological sample can comprise one or more cells from the subject. In some embodiments, a sample or biological sample can comprise one or more tissue samples from the subject. In some embodiments the sample is a biological sample.

The terms “body fluid” or “bodily fluids” are liquids originating from inside the bodies of organisms. Bodily fluids include amniotic fluid, aqueous humour, vitreous humour, bile, whole blood, blood, serum, plasma, breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. Extracellular bodily fluids include intravascular fluid (blood plasma), interstitial fluids, lymphatic fluid and transcellular fluid. Immunoglobulin G (IgG), the most abundant antibody subclass, may be found in all body fluids. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device. Additional sample collection procedures and devices include but are not limited to: a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS™) samples can be stored and mailed, and an assay can be performed remotely.

“Beneficial results” or “desired results” may include, but are in no way limited to, lessening or alleviating the severity of the disease, disorder, condition, medical condition, or disease condition, preventing the disease, disorder, condition, medical condition, or disease condition from worsening, curing the disease, disorder, condition, medical condition, or disease condition, preventing the disease, disorder, condition, medical condition, or disease condition from developing, lowering the chances of a patient developing the disease, disorder, condition, medical condition, or disease condition and prolonging a patient's life or life expectancy. Beneficial or desired clinical results include, but are not limited to, alleviation of one or more symptom(s), diminishment of extent of the deficit, stabilized (i.e., not worsening) state of disease, disorder, condition, medical condition, or disease condition progression, delay or slowing of metastasis or invasiveness, and amelioration or palliation of symptoms associated with the disease, disorder, condition, medical condition, or disease condition (e.g., cancer). Treatment also includes a decrease in mortality or an increase in the lifespan of a subject as compared to one not receiving the treatment.

As used herein, the term “administering,” refers to the placement of a compound or an agent (e.g., a drug, combination of drugs, therapeutic agent, pharmaceutical composition, FXR agonist, SAMe, existing therapy, etc.) or a treatment as disclosed herein into a subject by a method or route which results in at least partial localization of the compound, drug, combination of drugs, agent, pharmaceutical composition, therapeutic agent, FXR agonist, SAMe, existing therapy, or treatment at a desired site.

“Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition, drug, combination of drugs, pharmaceutical composition, treatment, therapeutic agent, FXR agonist, SAMe, or existing therapy, as disclosed herein.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. The term does not denote a particular age or sex. Thus, adult and newborn subjects, as well as fetuses, whether male or female, are intended to be included within the scope of this term. In some embodiments, the subject is a human.

In various embodiments, a subject can be one who has been previously diagnosed with or identified as suffering from or having a disease, disorder, condition, medical condition, or disease condition in need of treatment or one or more complications related to the disease, disorder, condition, medical condition, or disease condition and optionally, have already undergone treatment for the disease, disorder, condition, medical condition, or disease condition or the one or more complications related to the disease, disorder, condition, medical condition, or disease condition. Alternatively, a subject can also be one who has not been previously diagnosed as having a disease, disorder, condition, medical condition, or disease condition or one or more complications related to the disease, disorder, condition, medical condition, or disease condition. For example, a subject can be one who exhibits one or more risk factors for a disease, disorder, condition, medical condition, or disease condition or one or more complications related to the disease, disorder, condition, medical condition, or disease condition or a subject who does not exhibit risk factors. For example, a subject can be one who exhibits one or more symptoms for a disease, disorder, condition, medical condition, or disease condition or one or more complications related to the disease, disorder, condition, medical condition, or disease condition or a subject who does not exhibit symptoms. A “subject in need” of diagnosis or treatment for a particular disease, disorder, condition, medical condition, or disease condition can be a subject suspected of having that disease, disorder, condition, medical condition, or disease condition; diagnosed as having that disease, disorder, condition, medical condition, or disease condition; already treated or being treated for that disease, disorder, condition, medical condition, or disease condition; not treated for that disease, disorder, condition, medical condition, or disease condition; or at risk of developing that disease, disorder, condition, medical condition, or disease condition.

In some embodiments, the subject may be a subject that has liver disease and has been administered at least one FXR agonist.

“Mammal,” as used herein, refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adult and newborn subjects, whether male or female, are intended to be included within the scope of this term. Unless otherwise indicated, the subjects described herein can include mammals.

In some embodiments, the subject is selected from the group consisting of a subject suspected of having liver disease, a subject that has liver disease, a subject diagnosed with liver disease, a subject that is at risk of developing liver disease, a subject that has been treated for liver disease, and a subject that is being treated for liver disease. In some embodiments, the subject is suspected of having liver disease. In some embodiments, the subject has liver disease. In some embodiments, the subject has been diagnosed with liver disease. In some embodiments, the subject is at risk of developing liver disease. In some embodiments, the subject has been treated for liver disease. In some embodiments, the subject is being treated for liver disease.

In some embodiments, the subject is suspected of having liver disease and has been or is being treated with at least one FXR agonist, wherein the subject also has a side-effect associated with or resulting from the FXR agonist and/or treatment with a FXR agonist.

In some embodiments, the subject has liver disease and has been or is being treated with at least one FXR agonist, wherein the subject also has a side-effect associated with or resulting from the FXR agonist and/or treatment with a FXR agonist.

In some embodiments, the subject has been diagnosed with liver disease and has been or is being treated with at least one FXR agonist, wherein the subject also has a side-effect associated with or resulting from the FXR agonist and/or treatment with a FXR agonist.

In some embodiments, the subject is at risk of developing liver disease and has been or is being treated with at least one FXR agonist, wherein the subject also has a side-effect associated with or resulting from the FXR agonist and/or treatment with a FXR agonist.

By “at risk of” is intended to mean at increased risk of, compared to a normal subject, or compared to a control group, e.g. a patient population. “Increased risk” or “elevated risk” mean any statistically significant increase in the probability, e.g., that the subject has the disease, disorder, condition, medical condition, or disease condition. In some embodiments, the risk is increased by at least 10%. In some embodiments, the risk is increased by at least 20%. In some embodiments, the risk is increased by at least 50% over the control group with which the comparison is being made.

The term “statistically significant” or “significantly” refers to statistical evidence that there is a difference. It is defined as the probability of making a decision to reject the null hypothesis when the null hypothesis is actually true. The decision is often made using the p-value.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease, disorder, condition, medical condition, or disease condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). The term “treating” includes reducing or alleviating at least one adverse effect or symptom of a disease, disorder, condition, medical condition, or disease condition, such as, for example a liver disease and/or a side-effect associated with or resulting from a FXR agonist.

“Therapeutically effective amount” as used herein refers to that amount which is capable of achieving beneficial results in a subject. A therapeutically effective amount can be determined on an individual basis and will be based, at least in part, on consideration of the physiological characteristics of the mammal, the type of delivery system or therapeutic technique used and the time of administration relative to the progression of the disease. In addition, “therapeutically effective amount” as used herein refers to that amount which is capable of achieving beneficial results in a subject with liver disease. A therapeutically effective amount can be determined on an individual basis and will be based, at least in part, on consideration of the physiological characteristics of the subject, the type of delivery system or therapeutic technique used and the time of administration relative to the progression of the disease, disorder, condition, medical condition, or disease condition.

“Therapeutic agents” as used herein refers to agents or compounds that are used to, for example, treat, inhibit, prevent, mitigate the effects of, reduce the severity of, reduce the likelihood of developing, slow the progression of a disease, disorder, condition, medical condition, or disease condition. Diseases targeted by the therapeutic agents include but are not limited to liver diseases including NASH, cholestatic liver diseases, alcoholic liver diseases, liver fibrosis, primary biliary cholangitis and/or pruritus. In some embodiments, the diseases targeted by the therapeutic agents include but are not limited to liver diseases including NASH, NAFLD, cholestatic liver disease, alcoholic liver disease, liver fibrosis, primary biliary cholangitis, pruritus or a combination thereof. In some embodiments, the therapeutic agent comprises at least one FXR agonist. In some embodiments, the therapeutic agent comprises SAMe. In some embodiments, the therapeutic agent comprises at least one FXR agonist and SAMe.

A “pharmaceutically acceptable salt”, as used herein, is intended to encompass any compound described herein that is utilized in the form of a salt thereof, especially where the salt confers on the compound improved pharmacokinetic properties as compared to the free form of compound or a different salt form of the compound. The pharmaceutically acceptable salt form can also initially confer desirable pharmacokinetic properties on the compound that it did not previously possess, and may even positively affect the pharmacodynamics of the compound with respect to its therapeutic activity in the body. An example of a pharmacokinetic property that can be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Pharmaceutically acceptable salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety. Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Suitable acids which are capable of forming salts with the compounds of the disclosure include inorganic acids such as hydrochloric acid, hydrobromic acid, perchloric acid, nitric acid, thiocyanic acid, sulfuric acid, phosphoric acid, and the like; and organic acids such as 1,2-ethanedisulfonic acid, 2-hydroxyethanesulfonic acid, 2-naphthalenesulfonic acid, 3-phenylpropionic acid, 4-methylbicyclo[2.2.2]oct-2-ene-1-carboxylic acid, 4,4′-mefhylenebis(3-hydroxy-2-ene-1-carboxylic acid), acetic acid, anthranilic acid, benzenesulfonic acid, benzoic acid, camphorsulfonic acid, cinnamic acid, citric acid, cyclopentanepropionic acid, ethanesulfonic acid, formic acid, fumaric acid, glucoheptonic acid, gluconic acid, glutamic acid, glycolic acid, heptanoic acid, hydroxynaphthoic acid, lactic acid, lauryl sulfuric acid, maleic acid, malic acid, malonic acid, mandelic acid, methanesulfonic acid, muconic acid, naphthalene sulfonic acid, o-(4-hydroxybenzoyl)benzoic acid, oxalic acid, p-chlorobenzenesulfonic acid, propionic acid, p-toluenesulfonic acid, pyruvic acid, salicylic acid, stearic acid, succinic acid, sulfanilic acid, tartaric acid, tertiary butylacetic acid, trifluoroacetic acid, trimethylacetic acid, and the like. Suitable bases capable of forming salts with the compounds of the disclosure include inorganic bases such as sodium hydroxide, ammonium hydroxide, sodium carbonate, calcium hydroxide, potassium hydroxide and the like; and organic bases such as mono-, di- and tri-alkyl and aryl amines (e.g., triethylamine, diisopropyl amine, methyl amine, dimethyl amine, N-methylglucamine, pyridine, picoline, dicyclohexylamine, N,N′-dibezylethylenediamine, and the like), and optionally substituted ethanol-amines (e.g., ethanolamine, diethanolamine, trierhanolamine and the like).

A used herein, the term “prodrug” refers to agents or compounds that are rendered less active by some moiety (e.g., a chemical or biological moiety) that can be converted via some chemical or physiological process (e.g., enzymatic processes and metabolic hydrolysis) to a therapeutic agent or active compound. Thus, the term “prodrug” also refers to a precursor of a biologically active compound that is pharmaceutically acceptable. A prodrug may be inactive when administered to a subject, e.g., an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “disease” refers to an abnormal condition affecting the body of an organism. The term “disorder” refers to a functional abnormality or disturbance. The term “condition” as used herein refers generally to a disease, disorder, event, or change in health status. The term “medical condition” includes, but is not limited to, any condition, disease, or disorder manifested as one or more physical and/or psychological symptoms for which treatment and/or prevention is desirable, and includes previously and newly identified condition, disease, or disorder. The term “disease condition” refers to an abnormal condition affecting the body of an organism that may be caused by a disease.

A “healthy subject” or “normal subject” is a subject that does not have a disease, disorder, condition, medical condition, or disease condition.

The term “unhealthy subject” or “abnormal subject” is a subject that does have a disease, disorder, condition, medical condition, or disease condition.

In various embodiments, the disease is liver disease. In various embodiments, the disorder is liver disease. In various embodiments, the condition is liver disease. In various embodiments, the medical condition is liver disease. In various embodiments, the disease condition is liver disease.

In various embodiments, the disease is a side-effect associated with or resulting from a FXR agonist and/or treatment with a FXR agonist. In various embodiments, the condition is a side-effect associated with or resulting from a FXR agonist and/or treatment with a FXR agonist. In various embodiments, the medical condition is a side-effect associated with or resulting from a FXR agonist and/or treatment with a FXR agonist. In various embodiments, the disease condition is a side-effect associated with or resulting from a FXR agonist and/or treatment with a FXR agonist.

In various embodiments, the disease comprises liver disease and a side-effect associated with or resulting from a FXR agonist and/or treatment with a FXR agonist. In various embodiments, the condition comprises liver disease and a side-effect associated with or resulting from a FXR agonist and/or treatment with a FXR agonist. In various embodiments, the medical condition comprises liver disease and a side-effect associated with or resulting from a FXR agonist and/or treatment with a FXR agonist. In various embodiments, the disease condition comprises liver disease and a side-effect associated with or resulting from a FXR agonist and/or treatment with a FXR agonist.

Non-limiting examples of liver disease include nonalcoholic fatty liver disease (NAFLD), nonalcoholic steatohepatitis (NASH), cholestatic liver disease, alcoholic liver disease, liver fibrosis, primary biliary cholangitis, pruritus, chronic hepatitis B, primary sclerosing cholangitis, or combinations thereof.

In various embodiments, the liver disease is selected from the group consisting of nonalcoholic fatty liver disease (NAFLD), nonalcoholic steatohepatitis (NASH), cholestatic liver disease, alcoholic liver disease, liver fibrosis, primary biliary cholangitis, pruritus, chronic hepatitis B, primary sclerosing cholangitis and combinations thereof.

Obeticholic acid (OCA) induces expression of the oncogene MAFG and can enhance liver cancer growth. The inventors found that S-adenosylmethionine (SAMe) decreases expression of MAFG. Without being bound to a particular theory, the inventors hypothesize that since OCA induces expression of MAFG and SAMe decreases expression of MAFG, combination of OCA and SAMe would provide a therapeutic benefit wherein OCA exerts therapeutic benefit in treating liver diseases and SAMe inhibits the side-effects of OCA.

Furthermore, SAMe may also offer additional therapeutic benefits. SAMe has been studied in a variety of liver injuries and cancers in animal models and found to be efficacious in 1) preventing the progression of NAFLD to NASH, 2) progression of liver fibrosis, 3) cholestatic liver injuries, 4) alcoholic liver injury, 5) endotoxemia-induced liver injury and 6) chemoprevention of liver cancer.

In addition, the inventors found that in various embodiments a combination of at least one FXR agonist and SAMe provide a therapeutic benefit, wherein the FXR agonist exerts therapeutic benefit in treating liver diseases and SAMe inhibits the side effects of the FXR agonist.

In some embodiments, a combination or mixture of at least one FXR agonist and SAMe provide a therapeutic benefit, wherein the combination or mixture of at least one FXR agonist and SAMe exerts therapeutic benefit in treating liver diseases, and a combination or mixture of at least one FXR agonist and SAMe inhibits the side effects of the FXR agonist.

S-adenosylmethionine (SAMe), CAS #: 29908-03-0.

In some embodiments, S-adenosylmethionine (SAMe) also includes any salts, esters, or prodrugs thereof. In some embodiments, the S-adenosylmethionine (SAMe) may optionally be in the form of any salts, esters, or prodrugs thereof. In some embodiments, S-adenosylmethionine (SAMe) also includes any pharmaceutically acceptable salts, esters, or prodrugs thereof. In some embodiments, the S-adenosylmethionine (SAMe) may optionally be in the form of any pharmaceutically acceptable salts, esters, or prodrugs thereof.

Farnesoid X Receptor (F) is a member of the nuclear receptor superfamily of ligand activated transcription factors (Lu et al. J. Biol. Chem. 2001 17:17). Farnesoid X receptor (FXR) is also known as NR1H4 (nuclear receptor subfamily 1, group H, member 4).

The term “FXR agonist” as used herein refers to an agent or compound that functions by targeting and selectively binding the farnesoid X receptor (FXR) and which activates or upregulates the activity of FXR.

In some embodiments, FXR agonist also includes any salts, esters, or prodrugs thereof. In some embodiments, any of the FXR agonists may optionally be in the form of any salts, esters, or prodrugs thereof. In some embodiments, FXR agonist also includes any pharmaceutically acceptable salts, esters, or prodrugs thereof. In some embodiments, any of the FXR agonists may optionally be in the form of any pharmaceutically acceptable salts, esters, or prodrugs thereof.

Non-limiting examples of FXR agonists include obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof.

In some embodiments, the at least one FXR agonist is selected from the group consisting of obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof.

In some embodiments, the at least one FXR agonist is selected from the group consisting of EDP-305, GS-9674, LMB-763, EYP-001, TERN-101, AGN-242266, EP-024297, M-480, MET-409, RDX-023, and combinations thereof.

In some embodiments, the at least one FXR agonist is selected from the group consisting of obeticholic acid (OCA), cholic acid, tropifexor, cafestol, fexaramine, GW4064, and combinations thereof.

In some embodiments, the at least one FXR agonist is selected from the group consisting of obeticholic acid (OCA), tropifexor, GW4064, and combinations thereof.

In some embodiments, the at least one FXR agonist is a bile acid, bile acid analog, or bile acid derivative, or any salts, esters, or prodrugs thereof. In some embodiments, the at least one FXR agonist is a bile acid, bile acid analog, or bile acid derivative, or any pharmaceutically acceptable salts, esters, or prodrugs thereof.

In some embodiments, the at least one bile acid is cholic acid, glycocholic acid, taurocholic acid, deoxycholic acid, chenodeoxycholic acid, glycochenodeoxycholic acid, taurochenodeoxycholic acid, lithocholic acid, or any salts, esters, or prodrugs thereof. In some embodiments, the at least one bile acid is cholic acid, glycocholic acid, taurocholic acid, deoxycholic acid, chenodeoxycholic acid, glycochenodeoxycholic acid, taurochenodeoxycholic acid, lithocholic acid, or any pharmaceutically acceptable salts, esters, or prodrugs thereof.

Obeticholic acid (OCA) (also known as 6α-ethyl-chenodeoxycholic acid, or INT-747), CAS #: 459789-99-2.

Cholic acid (also known as 3α,7α,12α-trihydroxy-5β-cholan-24-oic acid), CAS #: 81-25-4.

Glycocholic acid (also known as cholylglycine), CAS #:475-31-0.

Taurocholic acid (also known as cholaic acid, cholyltaurine, or acidum cholatauricum),

CAS #: 81-24-3.

Deoxycholic acid (also known as cholanoic acid, Kybella, Celluform Plus, Belkyra, and 3α,12α-dihydroxy-5β-cholan-24-oic acid), CAS #: 83-44-3.

Chenodeoxycholic acid (also known as chenodesoxycholic acid, chenocholic acid and 3α,7α-dihydroxy-5β-cholan-24-oic acid), CAS #: 474-25-9.

Glycochenodeoxycholic acid, CAS #: 640-79-9.

Taurochenodeoxycholic acid, CAS #: 516-35-8.

Lithocholic acid, also known as 3α-hydroxy-5β-cholan-24-oic acid or LCA, CAS #: 434-13-9.

Tropifexor (also known as LJN452), CAS #: 1383816-29-2.

Fexaramine, CAS #: 574013-66-4.

Cafestol, CAS #: 469-83-0.

GW4064 is described in US2007/0015796

EDP-305 is a FXR agonist manufactured by Enanta Pharmaceuticals, Inc. GS-9674 is a FXR agonist manufactured by Gilead Sciences Inc. LMB-763 is a FXR agonist manufactured by Novartis AG. EYP-001 is a FXR agonist manufactured by Enyo Pharma SA. TERN-101 is a FXR agonist manufactured by Terns Pharmaceuticals Inc. AGN-242266 is a FXR agonist manufactured by Allergan Plc. EP-024297 is a FXR agonist manufactured by Enanta Pharmaceuticals Inc. M-480 is a FXR agonist manufactured by Metacrine Inc. MET-409 is a FXR agonist manufactured by Metacrine Inc. RDX-023 manufactured by Ardelyx Inc.

Various Non-Limiting Embodiments of the Invention

Methods for Treating, Inhibiting, Reducing the Severity of and/or Promoting Prophylaxis of Liver Disease

Accordingly, provided herein are methods for treating, inhibiting, reducing the severity of and/or promoting prophylaxis of liver diseases in a subject in need thereof. The methods include administering a therapeutically effective amount of OCA and SAMe to the subject so as to treat, inhibit, reduce the severity of and/or promote prophylaxis of liver diseases in the subject. In one embodiment, OCA and SAMe are administered sequentially. In another embodiment, OCA and SAMe are administered simultaneously. In one embodiment, the subject is human. In some embodiments, the OCA and SAMe are administered with existing therapies for liver diseases.

Further, provided herein are methods for treating, inhibiting, and/or reducing the severity of non-alcoholic fatty liver disease (NAFLD) (including the more severe form call non-alcoholic steatohepatitis or NASH) in a subject in need thereof. The methods include administering a therapeutically effective amount of OCA and SAMe to the subject so as to treat, inhibit, and/or reduce the severity of NAFLD in the subject. In one embodiment, OCA and SAMe are administered sequentially. In another embodiment, OCA and SAMe are administered simultaneously. In one embodiment, the subject is human. In some embodiments, the OCA and SAMe are administered with existing therapies for NAFLD.

Also, provided herein are methods for treating, inhibiting, and/or reducing the severity of cholestatic liver disease in a subject in need thereof. The methods include administering a therapeutically effective amount of OCA and SAMe to the subject so as to treat, inhibit and/or reduce the severity of cholestatic liver disease in the subject. In one embodiment, the cholestatic liver disease is primary biliary cholangitis (PBC). In one embodiment, OCA and SAMe are administered sequentially. In another embodiment, OCA and SAMe are administered simultaneously. In one embodiment, the subject is human. In some embodiments, the OCA and SAMe are administered with existing therapies for cholestatic liver diseases.

Also, provided herein are methods for treating, inhibiting and/or reducing the severity of alcoholic liver disease in a subject in need thereof. The methods include administering a therapeutically effective amount of OCA and SAMe to the subject so as to treat, inhibit and/or reduce the severity of alcoholic liver disease in the subject. In one embodiment, OCA and SAMe are administered sequentially. In another embodiment, OCA and SAMe are administered simultaneously. In one embodiment, the subject is human. In some embodiments, the OCA and SAMe are administered with existing therapies for alcoholic liver diseases

Further, provided herein are methods for treating, inhibiting, reducing the severity of and/or promoting prophylaxis of pruritus in a subject in need thereof. The methods include administering a therapeutically effective amount of SAMe to the subject so as to treat, inhibit, reduce the severity of and/or promote prophylaxis of pruritus in the subject. In one embodiment, the pruritus is due to administration of OCA. Accordingly, in one embodiment, the subject has PBC, is being treated with OCA and has pruritus. In another embodiment, the subject has NAFLD, is being treated with OCA and has pruritus. In a further embodiment, the subject has alcoholic liver disease, is being treated with OCA and has pruritus. In one embodiment, SAMe is administered before, during and/or after administration of OCA. In one embodiment, the subject is human. In some embodiments, SAMe is administered with existing therapies for pruritus in subjects that are being treated or have been treated with OCA.

Also, provided herein are methods for treating, inhibiting, reducing the severity of and/or promoting prophylaxis of liver fibrosis in a subject in need thereof. The methods include administering a therapeutically effective amount of OCA and SAMe to the subject so as to treat, inhibit, reduce the severity of and/or promote prophylaxis of liver fibrosis in the subject. In one embodiment, OCA and SAMe are administered sequentially. In another embodiment, OCA and SAMe are administered simultaneously. In one embodiment, the subject is human. In some embodiments, the OCA and SAMe are administered with existing therapies for liver fibrosis.

Provided herein are methods for treating, inhibiting, reducing the severity of and/or promoting prophylaxis of disease-states, wherein the disease-state is treatable with OCA. The methods include administering a therapeutically effective amount of SAMe to the subject wherein the subject has undergone, is undergoing or will undergo treatment with OCA, so as to treat, inhibit, reduce the severity of and/or promote prophylaxis of disease-states, wherein the disease-state is treatable with OCA.

In various embodiments, the present invention provides methods for treating, inhibiting, reducing the severity of and/or promoting prophylaxis of liver diseases in a subject in need thereof. The methods include administering a therapeutically effective amount of at least one FXR agonist and SAMe to the subject so as to treat, inhibit, reduce the severity of and/or promote prophylaxis of liver diseases in the subject. In one embodiment, the at least one FXR agonist and SAMe are administered sequentially. In another embodiment, the at least one FXR agonist and SAMe are administered simultaneously. In one embodiment, the subject is human. In some embodiments, the at least one FXR agonist and SAMe are administered with existing therapies for liver diseases.

In various embodiments, the present invention provides methods for treating, inhibiting, and/or reducing the severity of non-alcoholic fatty liver disease (NAFLD) (including the more severe form call non-alcoholic steatohepatitis or NASH) in a subject in need thereof. The methods include administering a therapeutically effective amount of at least one FXR agonist and SAMe to the subject so as to treat, inhibit, and/or reduce the severity of NAFLD in the subject. In one embodiment, the at least one FXR agonist and SAMe are administered sequentially. In another embodiment, the at least one FXR agonist and SAMe are administered simultaneously. In one embodiment, the subject is human. In some embodiments, the at least one FXR agonist and SAMe are administered with existing therapies for NAFLD.

In various embodiments, the present invention provides methods for treating, inhibiting, and/or reducing the severity of cholestatic liver disease in a subject in need thereof. The methods include administering a therapeutically effective amount of at least one FXR agonist and SAMe to the subject so as to treat, inhibit and/or reduce the severity of cholestatic liver disease in the subject. In one embodiment, the cholestatic liver disease is primary biliary cholangitis (PBC). In one embodiment, the at least one FXR agonist and SAMe are administered sequentially. In another embodiment, the at least one FXR agonist and SAMe are administered simultaneously. In one embodiment, the subject is human. In some embodiments, the at least one FXR agonist and SAMe are administered with existing therapies for cholestatic liver diseases.

In various embodiments, the present invention provides methods for treating, inhibiting and/or reducing the severity of alcoholic liver disease in a subject in need thereof. The methods include administering a therapeutically effective amount of at least one FXR agonist and SAMe to the subject so as to treat, inhibit and/or reduce the severity of alcoholic liver disease in the subject. In one embodiment, the at least one FXR agonist and SAMe are administered sequentially. In another embodiment, the at least one FXR agonist and SAMe are administered simultaneously. In one embodiment, the subject is human. In some embodiments, the at least one FXR agonist and SAMe are administered with existing therapies for alcoholic liver diseases

In some embodiments, the present invention provides methods for treating, inhibiting, reducing the severity of and/or promoting prophylaxis of pruritus in a subject in need thereof. The methods include administering a therapeutically effective amount of SAMe to the subject so as to treat, inhibit, reduce the severity of and/or promote prophylaxis of pruritus in the subject. In one embodiment, the pruritus is due to administration of at least one FXR agonist. Accordingly, in one embodiment, the subject has PBC, is being treated with at least one FXR agonist and has pruritus. In another embodiment, the subject has NAFLD, is being treated with at least one FXR agonist and has pruritus. In a further embodiment, the subject has alcoholic liver disease, is being treated with at least one FXR agonist and has pruritus. In one embodiment, SAMe is administered before, during and/or after administration of the at least one FXR agonist. In one embodiment, the subject is human. In some embodiments, SAMe is administered with existing therapies for pruritus in subjects that are being treated or have been treated with at least one FXR agonist.

In various embodiments, the present invention provides methods for treating, inhibiting, reducing the severity of and/or promoting prophylaxis of liver fibrosis in a subject in need thereof. The methods include administering a therapeutically effective amount of at least one FXR agonist and SAMe to the subject so as to treat, inhibit, reduce the severity of and/or promote prophylaxis of liver fibrosis in the subject. In one embodiment, the at least one FXR agonist and SAMe are administered sequentially. In another embodiment, the at least one FXR agonist and SAMe are administered simultaneously. In one embodiment, the subject is human. In some embodiments, the at least one FXR agonist and SAMe are administered with existing therapies for liver fibrosis.

In various embodiments, the present invention provides methods for treating, inhibiting, reducing the severity of and/or promoting prophylaxis of disease-states, wherein the disease-state is treatable with at least one FXR agonist. The methods include administering a therapeutically effective amount of SAMe to the subject wherein the subject has undergone, is undergoing or will undergo treatment with at least one FXR agonist, so as to treat, inhibit, reduce the severity of and/or promote prophylaxis of disease-states, wherein the disease-state is treatable with at least one FXR agonist.

### Methods for Treating Liver Disease

In various embodiments, the present invention provides a method for treating liver disease in a subject, comprising: administering to the subject a therapeutically effective amount of at least one FXR agonist; and administering a therapeutically effective amount of SAMe to the subject, thereby treating liver disease in the subject.

In various embodiments, the present invention provides a method for treating liver disease in a subject, comprising: administering to the subject a therapeutically effective amount of at least one FXR agonist, and a therapeutically effective amount of SAMe, thereby treating liver disease in the subject.

In various embodiments, the present invention provides a method for treating liver disease in a subject, comprising: administering a treatment to the subject, wherein the treatment comprises a therapeutically effective amount of at least one FXR agonist and a therapeutically effective amount of SAMe.

In various embodiments, the present invention provides a method for treating liver disease in a subject, comprising: administering a treatment to the subject, wherein the treatment comprises a therapeutically effective amount of at least one FXR agonist and a therapeutically effective amount of SAMe, wherein the FXR agonist is administered to the subject to treat the liver disease, and the SAMe is administered to the subject to treat and/or inhibit and/or reduce a side-effect in the subject, wherein the side-effect is associated with or results from the FXR agonist or treatment with the FXR agonist.

In various embodiments, the present invention provides a method for treating liver disease in a subject, comprising: administering a treatment to the subject, wherein the treatment comprises a therapeutically effective amount of at least one FXR agonist and a therapeutically effective amount of SAMe, wherein the FXR agonist and SAMe treats the liver disease, and wherein the FXR agonist and SAMe treats, inhibits and/or reduces a side-effect in the subject, wherein the side-effect is associated with or results from the FXR agonist or treatment with the FXR agonist.

In some embodiments, the FXR agonist is administered to the subject to treat the liver disease, and the SAMe is administered to the subject to treat and/or inhibit and/or reduce a side-effect in the subject, wherein the side-effect is associated with or results from the FXR agonist or treatment with a FXR agonist. In some embodiments, the SAMe is administered to the subject to treat and/or inhibit and/or reduce a side-effect in the subject, wherein the side-effect is associated with or results from the FXR agonist or treatment with the FXR agonist. In some embodiments, the FXR agonist and SAMe are administered to the subject to treat the liver disease, and the FXR agonist and SAMe are administered to the subject to treat and/or inhibit and/or reduce a side-effect in the subject, wherein the side-effect is associated with or results from the FXR agonist or treatment with a FXR agonist.

In some embodiments, the FXR agonist treats the liver disease, and the SAMe treats and/or inhibits and/or reduces a side-effect in the subject, wherein the side-effect is associated with or results from the FXR agonist or treatment with a FXR agonist. In some embodiments, the SAMe treats and/or inhibits and/or reduces a side-effect in the subject, wherein the side-effect is associated with or results from the FXR agonist or treatment with the FXR agonist. In some embodiments, the FXR agonist and SAMe treat the liver disease, and the FXR agonist and SAMe treat and/or inhibit and/or reduce a side-effect in the subject, wherein the side-effect is associated with or results from the FXR agonist or treatment with a FXR agonist.

In some embodiments, the method further or optionally comprises prescribing at least one existing therapy to the subject. In some embodiments, the method further or optionally comprises administering at least one existing therapy to the subject. In some embodiments, the treatment further or optionally comprises a therapeutically effective amount of at least one existing therapy. In some embodiments, the treatment further or optionally comprises administering at least one existing therapy to the subject.

In some embodiments, the treatment comprises a combination or mixture of at least one FXR agonist and SAMe. In some embodiments, the treatment comprises a combination or mixture of a therapeutically effective amount of at least one FXR agonist and a therapeutically effective amount of SAMe.

In various embodiments, the present invention provides a method for treating liver disease in a subject, comprising: administering a treatment to the subject, wherein the treatment comprises a combination or mixture of a therapeutically effective amount of at least one FXR agonist and a therapeutically effective amount of SAMe. In various embodiments, the present invention provides a method for treating liver disease in a subject, comprising: administering a treatment to the subject, wherein the treatment comprises a combination or mixture of a therapeutically effective amount of at least one FXR agonist and a therapeutically effective amount of SAMe, wherein the combination or mixture of FXR agonist and SAMe treats the liver disease, and wherein the combination or mixture of FXR agonist and SAMe treats, inhibits and/or reduces a side-effect in the subject, wherein the side-effect is associated with or results from the FXR agonist or treatment with the FXR agonist.

In some embodiments, the treatment is a combination therapy. In some embodiments, the treatment is a combination treatment.

### Methods for Inhibiting Liver Disease

In various embodiments, the present invention provides a method for inhibiting liver disease in a subject, comprising: administering a treatment to the subject, wherein the treatment comprises a therapeutically effective amount of at least one FXR agonist and a therapeutically effective amount of SAMe.

In various embodiments, the present invention provides a method for inhibiting liver disease in a subject, comprising: administering to the subject a therapeutically effective amount of at least one FXR agonist; and administering a therapeutically effective amount of SAMe to the subject, thereby inhibiting liver disease in the subject.

In various embodiments, the present invention provides a method for inhibiting liver disease in a subject, comprising: administering to the subject a therapeutically effective amount of at least one FXR agonist, and a therapeutically effective amount of SAMe, thereby inhibiting liver disease in the subject.

In various embodiments, the present invention provides a method for inhibiting liver disease in a subject, comprising: administering a treatment to the subject, wherein the treatment comprises a therapeutically effective amount of at least one FXR agonist and a therapeutically effective amount of SAMe, wherein the FXR agonist is administered to the subject to inhibit the liver disease, and the SAMe is administered to the subject to treat and/or inhibit and/or reduce a side-effect in the subject, wherein the side-effect is associated with or results from the FXR agonist or treatment with the FXR agonist.

In various embodiments, the present invention provides a method for inhibiting liver disease in a subject, comprising: administering a treatment to the subject, wherein the treatment comprises a therapeutically effective amount of at least one FXR agonist and a therapeutically effective amount of SAMe, wherein the FXR agonist and SAMe inhibits the liver disease, and wherein the FXR agonist and SAMe treats, inhibits and/or reduces a side-effect in the subject, wherein the side-effect is associated with or results from the FXR agonist or treatment with the FXR agonist.

In some embodiments, the FXR agonist is administered to the subject to inhibit the liver disease, and the SAMe is administered to the subject to treat and/or inhibit and/or reduce a side-effect in the subject, wherein the side-effect is associated with or results from the FXR agonist or treatment with a FXR agonist. In some embodiments, the SAMe is administered to the subject to treat and/or inhibit and/or reduce a side-effect in the subject, wherein the side-effect is associated with or results from the FXR agonist or treatment with the FXR agonist. In some embodiments, the FXR agonist and SAMe are administered to the subject to inhibit the liver disease, and the FXR agonist and SAMe are administered to the subject to treat and/or inhibit and/or reduce a side-effect in the subject, wherein the side-effect is associated with or results from the FXR agonist or treatment with a FXR agonist.

In some embodiments, the FXR agonist inhibits the liver disease, and the SAMe treats and/or inhibits and/or reduces a side-effect in the subject, wherein the side-effect is associated with or results from the FXR agonist or treatment with a FXR agonist. In some embodiments, the SAMe treats and/or inhibits and/or reduces a side-effect in the subject, wherein the side-effect is associated with or results from the FXR agonist or treatment with the FXR agonist. In some embodiments, the FXR agonist and SAMe inhibits the liver disease, and the FXR agonist and SAMe treat and/or inhibit and/or reduce a side-effect in the subject, wherein the side-effect is associated with or results from the FXR agonist or treatment with a FXR agonist.

In some embodiments, the method further or optionally comprises prescribing at least one existing therapy to the subject. In some embodiments, the method further or optionally comprises administering at least one existing therapy to the subject. In some embodiments, the treatment further or optionally comprises a therapeutically effective amount of at least one existing therapy. In some embodiments, the treatment further or optionally comprises administering at least one existing therapy to the subject.

In various embodiments, the present invention provides a method for inhibiting liver disease in a subject, comprising: administering a treatment to the subject, wherein the treatment comprises a combination or mixture of a therapeutically effective amount of at least one FXR agonist and a therapeutically effective amount of SAMe. In various embodiments, the present invention provides a method for inhibiting liver disease in a subject, comprising: administering a treatment to the subject, wherein the treatment comprises a combination or mixture of a therapeutically effective amount of at least one FXR agonist and a therapeutically effective amount of SAMe, wherein the combination or mixture of FXR agonist and SAMe inhibits the liver disease, and wherein the combination or mixture of FXR agonist and SAMe treats, inhibits and/or reduces a side-effect in the subject, wherein the side-effect is associated with or results from the FXR agonist or treatment with the FXR agonist.

### Methods for Reducing the Severity of Liver Disease

In various embodiments, the present invention provides a method for reducing the severity of liver disease in a subject, comprising: administering a treatment to the subject, wherein the treatment comprises a therapeutically effective amount of at least one FXR agonist and a therapeutically effective amount of SAMe.

In various embodiments, the present invention provides a method for reducing the severity of liver disease in a subject, comprising: administering to the subject a therapeutically effective amount of at least one FXR agonist; and administering a therapeutically effective amount of SAMe to the subject, thereby reducing the severity of liver disease in the subject.

In various embodiments, the present invention provides a method for reducing the severity of liver disease in a subject, comprising: administering to the subject a therapeutically effective amount of at least one FXR agonist, and a therapeutically effective amount of SAMe, thereby reducing the severity of liver disease in the subject.

In various embodiments, the present invention provides a method for reducing the severity of liver disease in a subject, comprising: administering a treatment to the subject, wherein the treatment comprises a therapeutically effective amount of at least one FXR agonist and a therapeutically effective amount of SAMe, wherein the FXR agonist is administered to the subject to reducing the severity of the liver disease, and the SAMe is administered to the subject to treat and/or inhibit and/or reduce the severity of a side-effect in the subject, wherein the side-effect is associated with or results from the FXR agonist or treatment with the FXR agonist.

In various embodiments, the present invention provides a method for reducing the severity of liver disease in a subject, comprising: administering a treatment to the subject, wherein the treatment comprises a therapeutically effective amount of at least one FXR agonist and a therapeutically effective amount of SAMe, wherein the FXR agonist and SAMe inhibits the liver disease, and wherein the FXR agonist and SAMe treats, inhibits and/or reduces a side-effect in the subject, wherein the side-effect is associated with or results from the FXR agonist or treatment with the FXR agonist.

In some embodiments, the FXR agonist is administered to the subject to reduce the severity of the liver disease, and the SAMe is administered to the subject to treat and/or inhibit and/or reduce a side-effect in the subject, wherein the side-effect is associated with or results from the FXR agonist or treatment with a FXR agonist. In some embodiments, the SAMe is administered to the subject to treat and/or inhibit and/or reduce a side-effect in the subject, wherein the side-effect is associated with or results from the FXR agonist or treatment with the FXR agonist. In some embodiments, the FXR agonist and SAMe are administered to the subject to reduce the severity of the liver disease, and the FXR agonist and SAMe are administered to the subject to treat and/or inhibit and/or reduce a side-effect in the subject, wherein the side-effect is associated with or results from the FXR agonist or treatment with a FXR agonist.

In some embodiments, the FXR agonist reduces the severity of the liver disease, and the SAMe treats and/or inhibits and/or reduces a side-effect in the subject, wherein the side-effect is associated with or results from the FXR agonist or treatment with a FXR agonist. In some embodiments, the SAMe treats and/or inhibits and/or reduces a side-effect in the subject, wherein the side-effect is associated with or results from the FXR agonist or treatment with the FXR agonist. In some embodiments, the FXR agonist and SAMe reduce the severity of the liver disease, and the FXR agonist and SAMe treat and/or inhibit and/or reduce a side-effect in the subject, wherein the side-effect is associated with or results from the FXR agonist or treatment with a FXR agonist.

In some embodiments, the method further or optionally comprises prescribing at least one existing therapy to the subject. In some embodiments, the method further or optionally comprises administering at least one existing therapy to the subject. In some embodiments, the treatment further or optionally comprises a therapeutically effective amount of at least one existing therapy. In some embodiments, the treatment further or optionally comprises administering at least one existing therapy to the subject.

In various embodiments, the present invention provides a method for reducing the severity of liver disease in a subject, comprising: administering a treatment to the subject, wherein the treatment comprises a combination or mixture of a therapeutically effective amount of at least one FXR agonist and a therapeutically effective amount of SAMe. In various embodiments, the present invention provides a method for reducing the severity of liver disease in a subject, comprising: administering a treatment to the subject, wherein the treatment comprises a combination or mixture of a therapeutically effective amount of at least one FXR agonist and a therapeutically effective amount of SAMe, wherein the combination or mixture of FXR agonist and SAMe reduces the severity of the liver disease, and wherein the combination or mixture of FXR agonist and SAMe treats, inhibits and/or reduces a side-effect in the subject, wherein the side-effect is associated with or results from the FXR agonist or treatment with the FXR agonist.

### Methods for Promoting Prophylaxis of Liver Disease

In various embodiments, the present invention provides a method for promoting prophylaxis of liver disease in a subject, comprising: administering a treatment to the subject, wherein the treatment comprises a therapeutically effective amount of at least one FXR agonist and a therapeutically effective amount of SAMe.

In various embodiments, the present invention provides a method for promoting prophylaxis of liver disease in a subject, comprising: administering to the subject a therapeutically effective amount of at least one FXR agonist; and administering a therapeutically effective amount of SAMe to the subject, thereby promoting prophylaxis of liver disease in the subject.

In various embodiments, the present invention provides a method for promoting prophylaxis of liver disease in a subject, comprising: administering to the subject a therapeutically effective amount of at least one FXR agonist, and a therapeutically effective amount of SAMe, thereby promoting prophylaxis of liver disease in the subject.

In various embodiments, the present invention provides a method for promoting prophylaxis of liver disease in a subject, comprising: administering a treatment to the subject, wherein the treatment comprises a therapeutically effective amount of at least one FXR agonist and a therapeutically effective amount of SAMe, wherein the FXR agonist is administered to the subject to promoting prophylaxis of the liver disease, and the SAMe is administered to the subject to treat and/or inhibit and/or reduce a side-effect in the subject, wherein the side-effect is associated with or results from the FXR agonist or treatment with the FXR agonist.

In various embodiments, the present invention provides a method for promoting prophylaxis of liver disease in a subject, comprising: administering a treatment to the subject, wherein the treatment comprises a therapeutically effective amount of at least one FXR agonist and a therapeutically effective amount of SAMe, wherein the FXR agonist and SAMe promoting prophylaxis the liver disease, and wherein the FXR agonist and SAMe treats, inhibits and/or reduces a side-effect in the subject, wherein the side-effect is associated with or results from the FXR agonist or treatment with the FXR agonist.

In some embodiments, the FXR agonist is administered to the subject to promote prophylaxis of the liver disease, and the SAMe is administered to the subject to treat and/or inhibit and/or reduce a side-effect in the subject, wherein the side-effect is associated with or results from the FXR agonist or treatment with a FXR agonist. In some embodiments, the SAMe is administered to the subject to treat and/or inhibit and/or reduce a side-effect in the subject, wherein the side-effect is associated with or results from the FXR agonist or treatment with the FXR agonist. In some embodiments, the FXR agonist and SAMe are administered to the subject to promote prophylaxis of the liver disease, and the FXR agonist and SAMe are administered to the subject to treat and/or inhibit and/or reduce a side-effect in the subject, wherein the side-effect is associated with or results from the FXR agonist or treatment with a FXR agonist.

In some embodiments, the FXR agonist promote prophylaxis of the liver disease, and the SAMe treats and/or inhibits and/or reduces a side-effect in the subject, wherein the side-effect is associated with or results from the FXR agonist or treatment with a FXR agonist. In some embodiments, the SAMe treats and/or inhibits and/or reduces a side-effect in the subject, wherein the side-effect is associated with or results from the FXR agonist or treatment with the FXR agonist. In some embodiments, the FXR agonist and SAMe promote prophylaxis of the liver disease, and the FXR agonist and SAMe treat and/or inhibit and/or reduce a side-effect in the subject, wherein the side-effect is associated with or results from the FXR agonist or treatment with a FXR agonist.

In some embodiments, the method further or optionally comprises prescribing at least one existing therapy to the subject. In some embodiments, the method further or optionally comprises administering at least one existing therapy to the subject. In some embodiments, the treatment further or optionally comprises a therapeutically effective amount of at least one existing therapy. In some embodiments, the treatment further or optionally comprises administering at least one existing therapy to the subject.

In various embodiments, the present invention provides a method for promoting prophylaxis of liver disease in a subject, comprising: administering a treatment to the subject, wherein the treatment comprises a combination or mixture of a therapeutically effective amount of at least one FXR agonist and a therapeutically effective amount of SAMe. In various embodiments, the present invention provides a method for promoting prophylaxis of liver disease in a subject, comprising: administering a treatment to the subject, wherein the treatment comprises a combination or mixture of a therapeutically effective amount of at least one FXR agonist and a therapeutically effective amount of SAMe, wherein the combination or mixture of FXR agonist and SAMe promotes prophylaxis of the liver disease, and wherein the combination or mixture of FXR agonist and SAMe treats, inhibits and/or reduces a side-effect in the subject, wherein the side-effect is associated with or results from the FXR agonist or treatment with the FXR agonist.

Methods for Treating, Reducing and/or Inhibiting Side-Effects of FXR Agonists

In various embodiments, the present invention provides a method for treating, reducing and/or inhibiting side-effects associated with therapeutic use of at least one FXR agonist in a subject, comprising: administering to the subject a therapeutically effective amount of at least one FXR agonist; and administering to the subject a therapeutically effective amount of SAMe.

In various embodiments, the present invention provides a method for treating, reducing and/or inhibiting a side-effect associated with therapeutic use of at least one FXR agonist in a subject, comprising: administering to the subject, a therapeutically effective amount of SAMe.

In various embodiments, the present invention provides a method for reducing a side-effect associated with or resulting from therapeutic use of at least one FXR agonist in a subject, comprising: administering to the subject a therapeutically effective amount of SAMe.

In various embodiments, the present invention provides a method for inhibiting a side-effect associated with or resulting from therapeutic use of at least one FXR agonist in a subject, comprising: administering to the subject a therapeutically effective amount of SAMe.

In various embodiments, the present invention provides a method for treating a side-effect associated with or resulting from the therapeutic use of at least one FXR agonist in a subject, comprising: administering to the subject a therapeutically effective amount of SAMe.

In various embodiments, the present invention provides a method for treating, inhibiting, and/or reducing a side-effect associated with or resulting from the therapeutic use of at least one FXR agonist in a subject, comprising: administering a treatment to the subject, wherein the treatment comprises a therapeutically effective amount of SAMe.

In various embodiments, the present invention provides a method for treating, reducing and/or inhibiting side-effects associated with therapeutic use of at least one FXR agonist in a subject, comprising: administering at treatment to the subject, wherein the treatment comprises a combination or mixture of at least one FXR agonist and SAMe. In some embodiments, the treatment comprises a combination or mixture of a therapeutically effective amount of at least one FXR agonist and a therapeutically effective amount of SAMe.

Non-limiting examples of side-effects associated with or resulting from the therapeutic use of at least one FXR agonist include liver cancer, enhanced liver cancer growth, and pruritus. In some embodiments, a side-effect associated with or resulting from the therapeutic use of at least one FXR agonist is selected from the group consisting of liver cancer, enhanced liver cancer growth, pruritus, and combinations thereof. Non-limiting examples of a side-effect associated with or resulting from a FXR agonist or treatment with a FXR agonist is selected from the group consisting of liver cancer, enhanced liver cancer growth, pruritus, and combinations thereof.

In some embodiments, the subject has been or is being treated with at least one FXR agonist. In some embodiments, the subject has been or is being administered at least one FXR agonist. In some embodiments, the subject has been treated with at least one FXR agonist, wherein the subject is suspected of having liver disease, has been diagnosed with liver disease, has liver disease, or is at risk of developing liver disease. In some embodiments, the subject is being treated with at least one FXR agonist, wherein the subject is suspected of having liver disease, has been diagnosed with liver disease, has liver disease, or is at risk of developing liver disease. In some embodiments, the subject has been administered at least one FXR agonist, wherein the subject is suspected of having liver disease, has been diagnosed with liver disease, has liver disease, or is at risk of developing liver disease. In some embodiments, the subject is being administered at least one FXR agonist, wherein the subject is suspected of having liver disease, has been diagnosed with liver disease, has liver disease, or is at risk of developing liver disease.

### Methods for Assessing the Efficacy of the Treatment

In various embodiments, the present invention provides a method for assessing the efficacy of the treatment, comprising comparing the side-effect in the subject to the side-effect in a control subject, wherein a decrease in the side-effect in the subject relative to the control subject is indicative of the efficacy of the treatment.

In various embodiments, the present invention provides a method for assessing the efficacy of the treatment, comprising comparing the liver disease in the subject to the liver disease in a control subject, wherein a decrease in the liver disease in the subject relative to the control subject is indicative of the efficacy of the treatment.

In various embodiments, the present invention provides a method for assessing the efficacy of the treatment, comprising comparing the liver disease and the side-effect in the subject to the liver disease and the side-effect in a control subject, wherein a decrease in the liver disease and the side-effect in the subject relative to the control subject is indicative of the efficacy of the treatment.

In various embodiments, the present invention provides a method for assessing the efficacy of the treatment, comprising comparing the presence of the side-effect in the subject to the presence of the side-effect in a control subject, wherein a decrease in the presence of the side-effect in the subject relative to the control subject is indicative of the efficacy of the treatment.

In various embodiments, the present invention provides a method for assessing the efficacy of the treatment, comprising comparing the presence of the liver disease in the subject to the presence of the liver disease in a control subject, wherein a decrease in the presence of the liver disease in the subject relative to the control subject is indicative of the efficacy of the treatment.

In various embodiments, the present invention provides a method for assessing the efficacy of the treatment, comprising comparing the presence of the liver disease and the side-effect in the subject to the presence of the liver disease and the side-effect in a control subject, wherein a decrease in the presence of the liver disease and the side-effect in the subject relative to the control subject is indicative of the efficacy of the treatment.

In various embodiments, the present invention provides a method for assessing the efficacy of the treatment, comprising comparing the presence of the side-effect in the subject to the presence of the side-effect in a control subject, wherein an absence of the side-effect in the subject relative to the control subject is indicative of the efficacy of the treatment.

In various embodiments, the present invention provides a method for assessing the efficacy of the treatment, comprising comparing the presence of the liver disease in the subject to the presence of the liver disease in a control subject, wherein an absence of the liver disease in the subject relative to the control subject is indicative of the efficacy of the treatment.

In various embodiments, the present invention provides a method for assessing the efficacy of the treatment, comprising comparing the presence of the liver disease and the side-effect in the subject to the presence of the liver disease and the side effect in a control subject, wherein an absence of the liver disease and the side-effect in the subject relative to the control subject is indicative of the efficacy of the treatment.

In various embodiments, the present invention provides a method for assessing the efficacy of the treatment, comprising comparing the severity of the side-effect in the subject to the severity of the side-effect in a control subject, wherein a decrease in the severity of the side-effect in the subject relative to the control subject is indicative of the efficacy of the treatment.

In various embodiments, the present invention provides a method for assessing the efficacy of the treatment, comprising comparing the severity of the liver disease in the subject to the severity of the liver disease in a control subject, wherein a decrease in the severity of the liver disease in the subject relative to the control subject is indicative of the efficacy of the treatment.

In various embodiments, the present invention provides a method for assessing the efficacy of the treatment, comprising comparing the severity of the liver disease and the side-effect in the subject to the severity of the liver disease and the side-effect in a control subject, wherein a decrease in the severity of the liver disease and the side-effect in the subject relative to the control subject is indicative of the efficacy of the treatment.

In some embodiments, the control subject has not been treated with the treatment. In some embodiments, the control subject has not been administered the treatment.

In some embodiments, the control subject is the subject at an earlier point in time. In some embodiments, the control subject is the patient at an earlier point in time.

In some embodiments, the control subject is the subject before being treated with the treatment. In some embodiments, the control subject is the patient before being treated with the treatment.

In some embodiments, the control subject is the subject before administration of the treatment. In some embodiments, the control subject is the patient before administration of the treatment.

In some embodiments, the control subject has liver disease. In some embodiments, the control subject has a side-effect associated with the FXR agonist. In some embodiments, the control subject has liver disease and a side-effect associated with or resulting from the FXR agonist. In some embodiments, the control subject has been treated with at least one FXR agonist, but has not been treated with SAMe. In some embodiments, the control subject has liver disease, has been treated with at least one FXR agonist, and has a side-effect associated with or resulting from the FXR agonist.

### Dosages

In exemplary embodiments, OCA and SAMe may be administered orally, intravenously, intramuscularly, intraperitoneally, or via inhalation.

In various embodiments, the therapeutically effective amount of OCA is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 10 to 50 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. Typical dosages of an effective amount of OCA can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample or the responses observed in the appropriate animal models. In various embodiments, OCA may be administered once a day (SID/QD), twice a day (BID), three times a day (TID), four times a day (QID), or more, so as to administer an effective amount of OCA to the subject, where the effective amount is any one or more of the doses described herein. It is to be understood that the ranges given here include all intermediate ranges.

In various embodiments, the therapeutically effective amount of OCA is any one or more of about 0.01 to 0.05 μg/kg, 0.05-0.1 μg/kg, 0.1 to 0.5 μg/kg, 0.5 to 5 μg/kg, 5 to 10 μg/kg, 10 to 20 μg/kg, 10 to 50 μg/kg, 20 to 50 μg/kg, 50 to 100 μg/kg, 100 to 150 μg/kg, 150 to 200 μg/kg, 200 to 250 μg/kg, 250 to 300 μg/kg, 300 to 350 μg/kg, 350 to 400 μg/kg, 400 to 500 μg/kg, 500 to 600 μg/kg, 600 to 700 μg/kg, 700 to 800 μg/kg, 800 to 900 μg/kg, 900 to 1000 μg/kg, 0.01 to 0.05 mg/kg, 0.05-0.1 mg/kg, 0.1 to 0.5 mg/kg, 0.5 to 1 mg/kg, 1 to 5 mg/kg, 5 to 10 mg/kg, 10 to 15 mg/kg, 15 to 20 mg/kg, 20 to 50 mg/kg, 50 to 100 mg/kg, 100 to 200 mg/kg, 200 to 300 mg/kg, 300 to 400 mg/kg, 400 to 500 mg/kg, 500 to 600 mg/kg, 600 to 700 mg/kg, 700 to 800 mg/kg, 800 to 900 mg/kg, 900 to 1000 mg/kg or a combination thereof. It is to be understood that the ranges given here include all intermediate ranges.

In various embodiments, the therapeutically effective amount of SAMe is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. Typical dosages of an effective amount of SAMe can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, SAMe may be administered once a day (SID/QD), twice a day (BID), three times a day (TID), four times a day (QID), or more, so as to administer an effective amount of SAMe to the subject, where the effective amount is any one or more of the doses described herein. It is to be understood that the ranges given here include all intermediate ranges.

In various embodiments, the therapeutically effective amount of SAMe is any one or more of about 0.01 to 0.05 μg/kg, 0.05-0.1 μg/kg, 0.1 to 0.5 μg/kg, 0.5 to 5 μg/kg, 5 to 10 μg/kg, 10 to 20 μg/kg, 10 to 50 μg/kg, 20 to 50 μg/kg, 50 to 100 μg/kg, 100 to 150 μg/kg, 150 to 200 μg/kg, 200 to 250 μg/kg, 250 to 300 μg/kg, 300 to 350 μg/kg, 350 to 400 μg/kg, 400 to 500 μg/kg, 500 to 600 μg/kg, 600 to 700 μg/kg, 700 to 800 μg/kg, 800 to 900 μg/kg, 900 to 1000 μg/kg, 0.01 to 0.05 mg/kg, 0.05-0.1 mg/kg, 0.1 to 0.5 mg/kg, 0.5 to 1 mg/kg, 1 to 5 mg/kg, 5 to 10 mg/kg, 10 to 15 mg/kg, 15 to 20 mg/kg, 20 to 50 mg/kg, 50 to 100 mg/kg, 100 to 200 mg/kg, 200 to 300 mg/kg, 300 to 400 mg/kg, 400 to 500 mg/kg, 500 to 600 mg/kg, 600 to 700 mg/kg, 700 to 800 mg/kg, 800 to 900 mg/kg, 900 to 1000 mg/kg or a combination thereof. It is to be understood that the ranges given here include all intermediate ranges.

In some embodiments, the therapeutically effective amount of SAMe is about 1-fold, about 2-fold, about 3-fold, about 4-fold, about 5-fold, about 6-fold, about 7-fold, about 8-fold, about 9-fold, about 10-fold, about 10-15 fold, about 15-20 fold, about 20-25 fold, about 25-30 fold, about 30-35 fold, about 35-40 fold, about 40-45 fold, about 45-50 fold, about 50-55 fold, about 55-60 fold, about 60-65 fold, about 65-70 fold, about 70-75 fold, about 75-80 fold, about 80-85 fold, about 85-90 fold, about 90-95 fold or about 95-100 fold higher than the effective amount of OCA.

In some embodiments, OCA and SAMe are administered simultaneously. In some embodiments, SAMe is administered before OCA. In some embodiments, SAMe is administered first followed by OCA followed by SAMe.

In exemplary embodiments, the at least one FXR agonist and SAMe may be administered orally, intravenously, intramuscularly, intraperitoneally, or via inhalation.

In various embodiments, the therapeutically effective amount of the at least one FXR agonist is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 10 to 50 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. Typical dosages of an effective amount of at least one FXR agonist can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample or the responses observed in the appropriate animal models. In various embodiments, the at least one FXR agonist may be administered once a day (SID/QD), twice a day (BID), three times a day (TID), four times a day (QID), or more, so as to administer an effective amount of the at least one FXR agonist to the subject, where the effective amount is any one or more of the doses described herein. It is to be understood that the ranges given here include all intermediate ranges.

In various embodiments, the therapeutically effective amount of the at least one FXR agonist is any one or more of about 0.01 to 0.05 μg/kg, 0.05-0.1 μg/kg, 0.1 to 0.5 μg/kg, 0.5 to 5 μg/kg, 5 to 10 μg/kg, 10 to 20 μg/kg, 10 to 50 μg/kg, 20 to 50 μg/kg, 50 to 100 μg/kg, 100 to 150 μg/kg, 150 to 200 μg/kg, 200 to 250 μg/kg, 250 to 300 μg/kg, 300 to 350 μg/kg, 350 to 400 μg/kg, 400 to 500 μg/kg, 500 to 600 μg/kg, 600 to 700 μg/kg, 700 to 800 μg/kg, 800 to 900 μg/kg, 900 to 1000 μg/kg, 0.01 to 0.05 mg/kg, 0.05-0.1 mg/kg, 0.1 to 0.5 mg/kg, 0.5 to 1 mg/kg, 1 to 5 mg/kg, 5 to 10 mg/kg, 10 to 15 mg/kg, 15 to 20 mg/kg, 20 to 50 mg/kg, 50 to 100 mg/kg, 100 to 200 mg/kg, 200 to 300 mg/kg, 300 to 400 mg/kg, 400 to 500 mg/kg, 500 to 600 mg/kg, 600 to 700 mg/kg, 700 to 800 mg/kg, 800 to 900 mg/kg, 900 to 1000 mg/kg or a combination thereof. It is to be understood that the ranges given here include all intermediate ranges.

In some embodiments, the therapeutically effective amount of SAMe is about 1-fold, about 2-fold, about 3-fold, about 4-fold, about 5-fold, about 6-fold, about 7-fold, about 8-fold, about 9-fold, about 10-fold, about 10-15 fold, about 15-20 fold, about 20-25 fold, about 25-30 fold, about 30-35 fold, about 35-40 fold, about 40-45 fold, about 45-50 fold, about 50-55 fold, about 55-60 fold, about 60-65 fold, about 65-70 fold, about 70-75 fold, about 75-80 fold, about 80-85 fold, about 85-90 fold, about 90-95 fold or about 95-100 fold higher than the effective amount of the at least one FXR agonist.

In some embodiments, the at least one FXR agonist and SAMe are administered simultaneously. In some embodiments, SAMe is administered before the at least one FXR agonist. In some embodiments, SAMe is administered first followed by at least one FXR agonist followed by SAMe. In some embodiments, the at least one FXR agonist is administered before the SAMe. In some embodiments, the at least one FXR agonist and SAMe are administered sequentially.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on the number of clinical factors. The desired dose can be administered every day, every other day, every third, fourth, fifth, or sixth day, etc. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Non-limiting examples of dosing schedules are administration daily, twice daily, three times daily or four or more times daily over a period of 1 week, 2 weeks, 3 weeks, 4 weeks, 1 month, 2 months, 3 months, 5 months, or 6 months or more.

### Compositions

In various embodiments, the present invention provides a composition comprising: SAMe.

In various embodiments, the present invention provides a composition comprising: an amount of SAMe.

In various embodiments, the present invention provides a composition comprising: a therapeutically effective amount of SAMe.

In various embodiments, the present invention provides a composition, comprising: at least one FXR agonist; and SAMe.

In various embodiments, the present invention provides a composition, comprising: an amount of at least one FXR agonist; and an amount of SAMe.

In various embodiments, the present invention provides a composition, comprising: a therapeutically effective amount of at least one FXR agonist; and a therapeutically effective amount of SAMe.

In some embodiments, the composition is for treating liver disease. In some embodiments, the composition is for treating liver disease, and for treating and/or inhibiting and/or reducing a side-effect associated with or resulting from the FXR agonist and/or treatment with a FXR agonist. In some embodiments, the composition is for treating and/or inhibiting and/or reducing a side-effect associated with or resulting from a FXR agonist and/or treatment with a FXR agonist.

In some embodiments, the composition is for treating, inhibiting, reducing the severity and/or promoting prophylaxis of liver disease. In some embodiments, the composition is for treating, inhibiting, reducing the severity and/or promoting prophylaxis of liver disease, and for treating, inhibiting, and/or reducing a side-effect associated with or resulting from the FXR agonist and/or treatment with a FXR agonist.

### Pharmaceutical Compositions

In various embodiments, the present invention provides a pharmaceutical composition comprising: SAMe.

In various embodiments, the present invention provides a pharmaceutical composition comprising: an amount of SAMe.

In various embodiments, the present invention provides a pharmaceutical composition comprising: a therapeutically effective amount of SAMe.

In various embodiments, the present invention provides a pharmaceutical composition, comprising: at least one FXR agonist; and SAMe.

In various embodiments, the present invention provides a pharmaceutical composition, comprising: an amount of at least one FXR agonist; and an amount of SAMe.

In various embodiments, the present invention provides a pharmaceutical composition, comprising: a therapeutically effective amount of at least one FXR agonist; and a therapeutically effective amount of SAMe.

In some embodiments, the pharmaceutical composition is for treating liver disease. In some embodiments, the pharmaceutical composition is for treating liver disease, and for treating and/or inhibiting and/or reducing a side-effect associated with or resulting from the FXR agonist and/or treatment with a FXR agonist. In some embodiments, the pharmaceutical composition is for treating and/or inhibiting and/or reducing a side-effect associated with or resulting from a FXR agonist and/or treatment with a FXR agonist.

In some embodiments, the pharmaceutical composition is for treating, inhibiting, reducing the severity and/or promoting prophylaxis of liver disease. In some embodiments, the pharmaceutical composition is for treating, inhibiting, reducing the severity and/or promoting prophylaxis of liver disease, and for treating, inhibiting, and/or reducing a side-effect associated with or resulting from the FXR agonist and/or treatment with a FXR agonist.

In various embodiments, the pharmaceutic al compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art. In certain embodiments, the pharmaceutical composition is formulated for intravascular, intravenous, intraarterial, intratumoral, intramuscular, subcutaneous, intranasal, intraperitoneal, or oral administration.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

In some embodiments, a buffer may be used in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

### Kits

In various embodiments, the present invention provides a kit for treating, inhibiting, reducing the severity of, and/or promoting prophylaxis of a disease, disorder, condition, medical condition, or disease condition in a subject. In some embodiments, the kit comprises: at least one FXR agonist; SAMe; and instructions for using the kit to treat a disease, disorder, condition, medical condition, or disease condition in a subject. In some embodiments, the kit comprises: a quantity of at least one FXR agonist; a quantity of SAMe; and instructions for using the kit to treat a disease, disorder, condition, medical condition, or disease condition in a subject. In some embodiments, the kit comprises: a therapeutically effective amount of at least one FXR agonist; a therapeutically effective amount of SAMe; and instructions for using the kit to treat a disease, disorder, condition, medical condition, or disease condition in a subject.

In various embodiments, the present invention provides a kit for treating, inhibiting, reducing the severity of, and/or promoting prophylaxis of liver disease in a subject, the kit comprising: a therapeutically effective amount of at least one FXR agonist; a therapeutically effective amount of SAMe; and instructions for using the kit to treat the liver disease in the subject.

In various embodiments, the present invention provides a kit for treating, inhibiting, reducing the severity of, and/or promoting prophylaxis of liver disease and for treating and/or inhibiting and/or reducing a side-effect associated with or resulting from the liver disease treatment, the kit comprising: a therapeutically effective amount of at least one FXR agonist; a therapeutically effective amount of SAMe; and instructions for using the kit to treat, inhibit, reduce the severity of, and/or promote prophylaxis of the liver disease and treat, inhibit, and/or reduce the side-effect.

In various embodiments, the present invention provides a kit for treating, inhibiting, and/or reducing a side-effect associated with or resulting from the therapeutic use of at least one FXR agonist, the kit comprising: a therapeutically effective amount of SAMe; and instructions for using the kit to treat, inhibit, and/or reduce the side-effect.

The kit is an assemblage of materials or components, including at least one FXR agonist and SAMe. The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

### Existing Therapies

In various embodiments, existing therapies for liver disease include are not limited to treatment with vitamin E, pioglitazone and/or life style changes including diet, exercise and weight loss, ursodeoxycholic acid, phenobarbital, cholestyramine, life style changes including diets rich in medium-chain triglycerides, long-chain triglycerides and/or treatment with oral absorbable, fat-soluble vitamin formulation A, D, E, and K supplementation, abstinence from alcohol, cessation of smoking, weight loss and/or treatment with steroids, Naltrexone, Acamprosate, Disulfiram, Topiramate and/or baclofen, eliminating hepatitis B virus or hepatitis C virus in chronic viral hepatitis, abstaining from alcohol, removing heavy metals such as iron in hemochromatosis or copper in Wilson disease, decompressing bile ducts in biliary obstruction and/or treatment with corticosteroids, penicillamine and/or colchicine, Ursodeoxycholic acid (UDCA), liver transplant, treatment with immunosuppressant drugs including methotrexate and/or colchicine, and/or treatment with fenofibrate and/or bezafibrate, and coffee.

In various embodiments, existing therapies for NAFLD and/or NASH include but are not limited to treatment with vitamin E, pioglitazone and/or life style changes including diet, exercise and weight loss. Therapeutically effective amounts of the existing therapies will be apparent to a person of skill in the art.

In various embodiments, existing therapies for cholestatic liver diseases include but are not limited to treatment with ursodeoxycholic acid, phenobarbital, cholestyramine, life style changes including diets rich in medium-chain triglycerides, long-chain triglycerides and/or treatment with oral absorbable, fat-soluble vitamin formulation A, D, E, and K supplementation. Therapeutically effective amounts of the existing therapies will be apparent to a person of skill in the art.

In various embodiments, existing therapies for alcoholic liver diseases include but are not limited to abstinence from alcohol, cessation of smoking, weight loss and/or treatment with steroids, Naltrexone, Acamprosate, Disulfiram, Topiramate and/or baclofen. Therapeutically effective amounts of the existing therapies will be apparent to a person of skill in the art.

In various embodiments, existing therapies for liver fibrosis include but are not limited to eliminating hepatitis B virus or hepatitis C virus in chronic viral hepatitis, abstaining from alcohol, removing heavy metals such as iron in hemochromatosis or copper in Wilson disease, decompressing bile ducts in biliary obstruction and/or treatment with corticosteroids, penicillamine and/or colchicine. Therapeutically effective amounts of the existing therapies will be apparent to a person of skill in the art.

In various embodiments, existing therapies for primary biliary cholangitis include but are not limited to treatment with Ursodeoxycholic acid (UDCA), obeticholic acid (OCA), liver transplant, treatment with immunosuppressant drugs including methotrexate and/or colchicine and/or treatment with fenofibrate and/or bezafibrate. Therapeutically effective amounts of the existing therapies will be apparent to a person of skill in the art.

In various embodiments, existing therapies for pruritus include but are not limited to treatment with antihistamines, cholestyramine, rifampin, opioid antagonists, Ondansetron, Ursodeoxycholic acid, Stanozolol, thalidomide, infused propofol, serotonin-selective reuptake inhibitors, UV-B, phenobarbital and/or dronabinol. Therapeutically effective amounts of the existing therapies will be apparent to a person of skill in the art.

In various embodiments, an existing therapy for liver disease is an additional therapy for liver disease. In various embodiments, an existing therapy is an additional therapy.

Some embodiments of the present invention can be defined as any of the following numbered paragraphs:

1. A method for treating liver disease in subject in need thereof, comprising: administering to the subject, a therapeutically effective amount of OCA and a therapeutically effective amount of SAMe.
2. The method of paragraph 1, wherein the liver disease is any one or more of NAFLD, cholestatic liver disease, alcoholic liver disease. liver fibrosis, primary biliary cholangitis, pruritus or a combination thereof.
3. The method of paragraph 1, wherein the OCA and SAMe are administered orally.
4. The method of paragraph 1, wherein the subject is human.
5. The method of paragraph 1, wherein the therapeutically effective amount of OCA is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.
6. The method of paragraph 1, wherein the therapeutically effective amount of SAMe is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day, about 1 gm per day to about 2 gm per day.
7. The method of paragraph 1, wherein SAMe is administered to the subject at a dose of about 400 mg three, four or five times per day or at a dose of 500 mg three or four times per day.
8. The method of paragraph 1, wherein OCA is administered to the subject at a dose of about 5 mg to 10 mg daily.
9. The method of paragraph 1, wherein the SAMe is administered at about the same, 1-fold, 5-fold, or 10-fold higher concentrations than OCA.
10. The method of paragraph 1, wherein OCA and SAMe are administered sequentially.
11. The method of paragraph 1, wherein OCA and SAMe are administered simultaneously.
12. The method of paragraph 1, wherein SAMe is administered before and after administration of SAMe.
12a. The method of paragraph 1, wherein SAMe is administered before or after administration of OCA.
13. The method of paragraph 1, wherein the OCA and SAMe are administered before, during and/or after the subject develops the liver disease.
14. The method of paragraph 1, wherein the OCA and SAMe are administrated to the subject 1-3 times per day or 1-7 times per week.
15. The method of paragraph 1, wherein the OCA and SAMe are administered to the subject for 1-5 days, 1-5 weeks, 1-5 months, 1-5 years, 5-10 years or longer.
16. The method of paragraph 1, further comprising prescribing existing therapies for liver diseases.
17. A method for reducing and/or inhibiting the side-effects associated with therapeutic use of OCA comprising, administering to the subject, a therapeutically effective amount of SAMe, wherein OCA and SAMe are administered sequentially or simultaneously.

Some embodiments of the present invention can be defined as any of the following numbered paragraphs:

18. A method for treating liver disease in a subject in need thereof, comprising: administering to the subject, a therapeutically effective amount of at least one FXR agonist and a therapeutically effective amount of SAMe.
19. The method of paragraph 18, wherein the liver disease is selected from the group consisting of nonalcoholic fatty liver disease (NAFLD), nonalcoholic steatohepatitis (NASH), cholestatic liver disease, alcoholic liver disease, liver fibrosis, primary biliary cholangitis, pruritus, chronic hepatitis B, primary sclerosing cholangitis, and combinations thereof.
20. The method of paragraph 18, wherein the at least one FXR agonist and SAMe are administered orally.
21. The method of paragraph 18, wherein the at least one FXR agonist is selected from the group consisting of obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof.
22. The method of paragraph 18, wherein the at least one FXR agonist is selected from the group consisting of obeticholic acid (OCA), cholic acid, tropifexor, cafestol, fexaramine, GW4064, and combinations thereof.
23. The method of paragraph 18, wherein the at least one FXR agonist is selected from the group consisting of obeticholic acid (OCA), tropifexor, GW4064, and combinations thereof.
24. The method of paragraph 18, wherein the subject is human.
25. The method of paragraph 18, wherein the therapeutically effective amount of the at least one FXR agonist is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.
26. The method of paragraph 18, wherein the therapeutically effective amount of SAMe is about 0.1 to 0.5 mg/kg/day, 0.5 to 5 mg/kg/day, 5 to 10 mg/kg/day, 10 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day, about 1 gm per day to about 2 gm per day.
27. The method of paragraph 18, wherein SAMe is administered to the subject at a dose of about 400 mg three, four or five times per day or at a dose of 500 mg three or four times per day.
28. The method of paragraph 18, wherein the at least one FXR agonist is administered to the subject at a dose of about 5 mg to 10 mg daily.
29. The method of paragraph 18, wherein the SAMe is administered at about the same, 1-fold, 5-fold, or 10-fold higher concentrations than the at least one FXR agonist.
30. The method of paragraph 18, wherein the at least one FXR agonist and SAMe are administered sequentially.
31. The method of paragraph 18, wherein the at least one FXR agonist and SAMe are administered simultaneously.
32. The method of paragraph 18, wherein SAMe is administered before and after administration of at least one FXR agonist.
33. The method of paragraph 18, wherein the at least one FXR agonist and SAMe are administered before, during and/or after the subject develops the liver disease.
34. The method of paragraph 18, wherein the at least one FXR agonist and SAMe are administrated to the subject 1-3 times per day or 1-7 times per week.
35. The method of paragraph 18, wherein the at least one FXR agonist and SAMe are administered to the subject for 1-5 days, 1-5 weeks, 1-5 months, 1-5 years, 5-10 years or longer.
36. The method of paragraph 18, further comprising administering an existing therapy for liver disease to the subject.
37. The method of paragraph 36, wherein the existing therapy is selected from the group consisting of treatment with vitamin E, pioglitazone and/or life style changes including diet, exercise and weight loss, ursodeoxycholic acid, phenobarbital, cholestyramine, life style changes including diets rich in medium-chain triglycerides, long-chain triglycerides and/or treatment with oral absorbable, fat-soluble vitamin formulation A, D, E, and K supplementation, abstinence from alcohol, cessation of smoking, weight loss and/or treatment with steroids, Naltrexone, Acamprosate, Disulfiram, Topiramate and/or baclofen, eliminating hepatitis B virus or hepatitis C virus in chronic viral hepatitis, abstaining from alcohol, removing heavy metals such as iron in hemochromatosis or copper in Wilson disease, decompressing bile ducts in biliary obstruction and/or treatment with corticosteroids, penicillamine and/or colchicine, Ursodeoxycholic acid (UDCA), liver transplant, treatment with immunosuppressant drugs including methotrexate and/or colchicine and/or treatment with fenofibrate and/or bezafibrate, and combinations thereof.
38. The method of paragraph 18, further comprising treating and/or inhibiting and/or reducing a side-effect in the subject.
39. The method of paragraph 38, wherein the side-effect is associated with or results from the FXR agonist or treatment with the FXR agonist.
40. The method of paragraph 38, wherein the side-effect is selected from the group consisting of liver cancer, enhanced liver cancer growth, pruritus, and combinations thereof.
41. A method for assessing the efficacy of the treatment of paragraph 38, comprising comparing the severity of the side-effect in the subject to the severity of the side-effect in a control subject, wherein a decrease in the severity of the side-effect in the subject relative to the control subject is indicative of the efficacy of the treatment.
42. A method for assessing the efficacy of the treatment of paragraph 38, comprising comparing the liver disease and the side-effect in the subject to the liver disease and the side-effect in a control subject, wherein a decrease in the liver disease and the side-effect in the subject relative to the control subject is indicative of the efficacy of the treatment.
43. A method for treating, reducing and/or inhibiting a side-effect associated with therapeutic use of at least one FXR agonist in a subject, comprising: administering to the subject, a therapeutically effective amount of SAMe.
44. The method of paragraph 43, further comprising administering a therapeutically effective amount of at least one FXR agonist.
45. The method of paragraph 43 or paragraph 44, wherein the at least one FXR agonist and SAMe are administered sequentially or simultaneously.
46. A pharmaceutical composition, comprising at least one FXR agonist, and SAMe.
47. The pharmaceutical composition of paragraph 46, further comprising at least one pharmaceutically acceptable carrier.
48. The pharmaceutical composition of paragraph 47, further comprising at least one pharmaceutically acceptable excipient.
49. The pharmaceutical composition of paragraph 46, wherein the at least one FXR agonist is selected from the group consisting of obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof.
50. The pharmaceutical composition of paragraph 46, wherein the at least one FXR agonist is selected from the group consisting of obeticholic acid (OCA), cholic acid, tropifexor, cafestol, fexaramine, GW4064, and combinations thereof.
51. The pharmaceutical composition of paragraph 46, wherein the at least one FXR agonist is selected from the group consisting of obeticholic acid (OCA), tropifexor, GW4064, and combinations thereof.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention. The invention is further illustrated by the following examples which are intended to be purely exemplary of the invention, and which should not be construed as limiting the invention in any way. The following examples are illustrative only, and are not intended to limit, in any manner, any of the aspects described herein. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

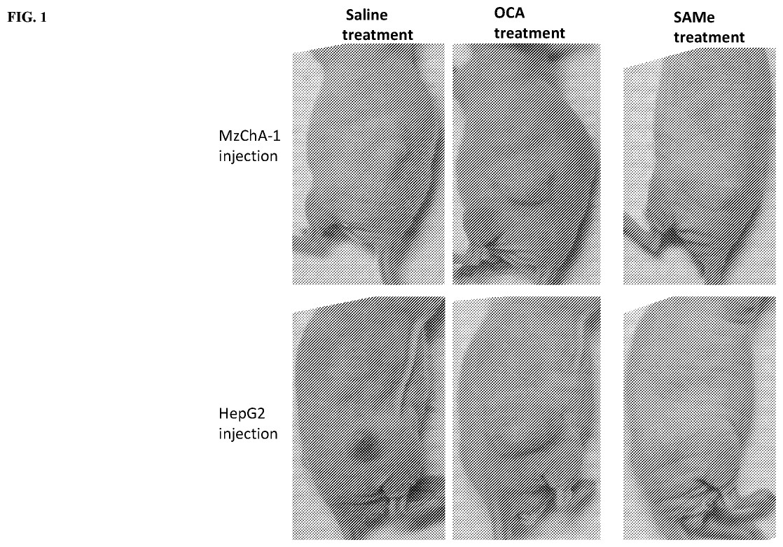

Mice were injected with MzChA-1 (cholangiocarcinoma) cells or HepG2 (immortalized cell line consisting of human liver carcinoma cells) cells. In mice treated with OCA, 30/mg/kg/day of OCA was administered orally. In mice treated with SAMe, 100 mg/kg/day was administered orally. FIG. 1 shows that in xenograft mice models tumor volumes in OCA group were at least 3-fold larger than that of SAMe group, which were lower than the control group.

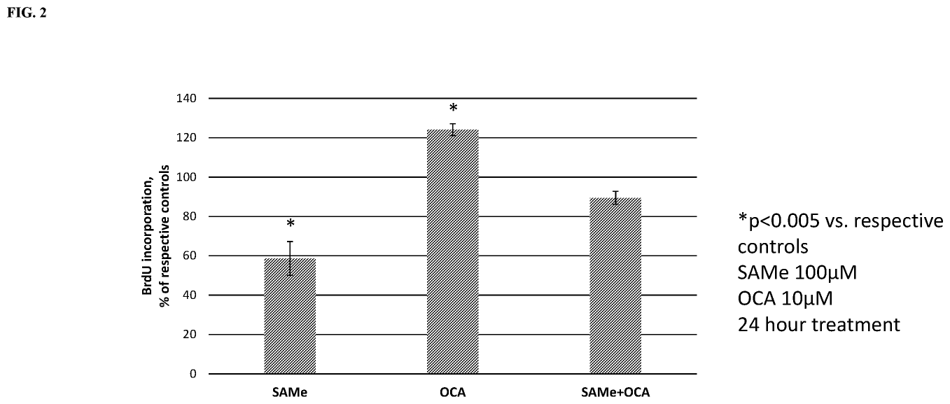

HepG2 cells were treated with 100 μM SAMe, 10 μM OCA individually or combined for 24 hours. Growth was determined using BrdU incorporation. SAMe was mixed in saline and OCA was mixed in DMSO (0.1% final), which yielded the same values. As shown in Table 1 and FIG. 2, while OCA alone induced growth of the cancer cell line, SAMe inhibited growth of the cancer cell line even in the presence of OCA. SAMe, OCA and SAMe+OCA were 58.65%, 124.05% and 89.5%, respectively, of their respective controls. These results show SAMe can block the growth-inductive effects of OCA.

TABLE 1
BrdU incorporation

| | HepG2 cell line | | | | | | | |
|---|---|---|---|---|---|---|---|---|
| | | | SAMe in | OCA in | | % of respective controls | | |
| | | Con | Saline | DMSO | SAMe + | | | SAMe + |
| | Con saline | DMSO | SAMe | OCA | OCA | SAMe | OCA | OCA |
| | 0.148 | 0.14 | 0.055 | 0.162 | 0.131 | | | |
| | 0.148 | 0.162 | 0.075 | 0.177 | 0.151 | | | |
| | 0.188 | 0.16 | 0.103 | 0.181 | 0.139 | | | |
| | 0.141 | 0.157 | 0.089 | 0.175 | 0.141 | | | |
| | 0.097 | 0.15 | 0.101 | 0.202 | 0.138 | | | |
| | 0.151 | 0.145 | 0.089 | 0.186 | 0.118 | | | |
| Ave | 0.15 | 0.15 | 0.09 | 0.18 | 0.14 | | | |
| | 0.03 | 0.01 | 0.02 | 0.01 | 0.01 | | | |
| | 0.01 | 0.00 | 0.01 | 0.01 | 0.00 | 58.65 | 124.05 | 89.50 |
| P< | Con Saline | | 0.001525 | | | | | |
| | vs. SAMe | | | | | | | |
| | Con DMSO | | | 0.001463 | | | | |
| | vs OCA | | | | | | | |
| | CON DMSO | | | | 0.019588 | | | |
| | vs. SAMe + | | | | | | | |
| | DMSO | | | | | | | |
| | SAMe vs OCA | | | 0.000001 | | | | |
| | SAMe vs. | | | | 0.000146 | | | |
| | SAMe + OCA | | | | | | | |
| | OCA vs. | | | | 0.000093 | | | |
| | SAMe + OCA | | | | | | | |
| | Saline vs. | | 0.593444 | | | | | |
| | DMSO | | | | | | | |

Notes:
Con is “control”.

### Example 2

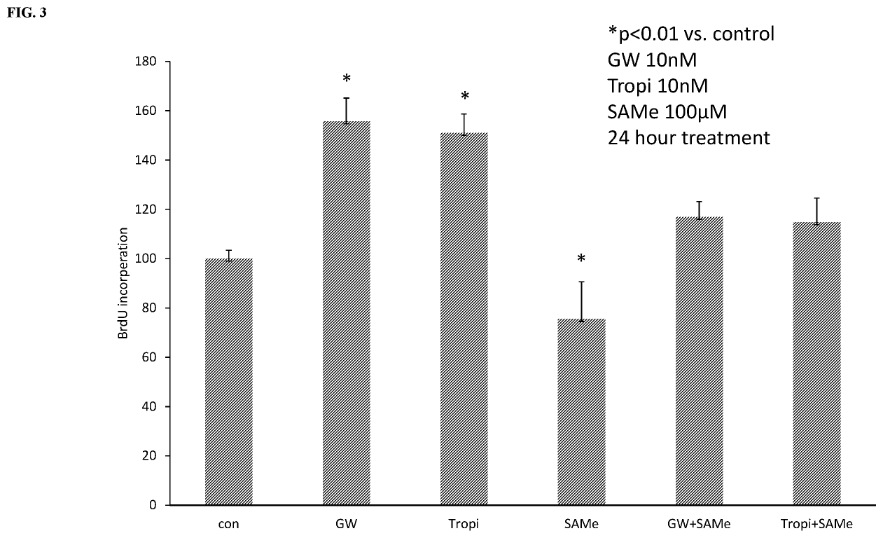

HepG2 (immortalized cell line consisting of human liver carcinoma cells) cells were treated with 100 μM SAMe, 10 nM GW4064 individually or combined for 24 hours. Growth was determined using BrdU incorporation. SAMe was mixed in saline and GW4064 was mixed in DMSO (0.1% final), which yielded the same values. As shown in Table 2 and FIG. 3, while GW4064 alone induced growth of the cancer cell line, SAMe inhibited growth of the cancer cell line even in the presence of GW4064. SAMe, GW4064 and SAMe+GW4064 were 75.52%, 155.70% and 116.97%, respectively, of their respective controls. These results show SAMe can block the growth-inductive effects of GW4064.

HepG2 (immortalized cell line consisting of human liver carcinoma cells) cells were treated with 100 μM SAMe, 10 nM Tropifexor individually or combined for 24 hours. Growth was determined using BrdU incorporation. SAMe was mixed in saline and Tropifexor was mixed in DMSO (0.1% final), which yielded the same values. As shown in Table 2 and FIG. 3, while Tropifexor alone induced growth of the cancer cell line, SAMe inhibited growth of the cancer cell line even in the presence of Tropifexor. SAMe, Tropifexor and SAMe+Tropifexor were 75.52%, 151.04% and 114.77%, respectively, of their respective controls. These results show SAMe can block the growth-inductive effects of Tropifexor.

TABLE 2
BrdU Incorporation

| | | | | | 10 nM + | 10 nM + |
|---|---|---|---|---|---|---|
| OD540 nm | | | | | 100 μM | 100 μM |
| HepG2 | | 10 nM | 10 nM | 100 μM | GW + | Tropi + |
| cell line | con | GW | Tropi | SAMe | SAMe | SAMe |
| | 0.091 | 0.128 | 0.173 | 0.113 | 0.096 | 0.131 |
| | 0.086 | 0.237 | 0.202 | 0.08 | 0.094 | 0.103 |
| | 0.097 | 0.136 | 0.153 | 0.065 | 0.12 | 0.14 |
| | 0.102 | 0.168 | 0.153 | 0.066 | 0.096 | 0.085 |
| | 0.097 | 0.164 | 0.121 | 0.064 | 0.125 | 0.088 |
| | 0.111 | 0.11 | 0.134 | 0.072 | 0.152 | 0.132 |
| | 0.104 | 0.128 | 0.125 | 0.059 | 0.114 | 0.146 |
| | 0.084 | 0.131 | 0.105 | 0.064 | 0.106 | 0.061 |
| AVE | 0.0965 | 0.15025 | 0.14575 | 0.072875 | 0.112875 | 0.11075 |
| % of | 100 | 155.6995 | 151.0363 | 75.51813 | 116.9689 | 114.7668 |
| respective | | | | | | |
| controls | | | | | | |
| p | | 0.00237 | 0.000776 | 0.004373 | 0.050999 | 0.23154 |
| SE | 3.374985 | 9.418074 | 7.590207 | 15.06151 | 6.154903 | 9.858348 |

| | | |
|---|---|---|
| P (test | 0.032636091 | 0.040915025 |
| versus | GW vs. GW + SAMe | Tropi vs. Tropi + SAMe |
| control) | | |

Notes:
Con is “control”; GW is “GW4064”; Tropi is “Tropifexor”.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Various embodiments of this application are described herein, including the best mode known to the inventors for carrying out the application. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method for treating liver disease in a subject in need thereof, comprising:
administering to the subject, a therapeutically effective amount of at least one FXR agonist and a therapeutically effective amount of SAMe.

2. The method of claim 1, wherein the liver disease is selected from the group consisting of nonalcoholic fatty liver disease (NAFLD), nonalcoholic steatohepatitis (NASH), cholestatic liver disease, alcoholic liver disease, liver fibrosis, primary biliary cholangitis, pruritus, chronic hepatitis B, primary sclerosing cholangitis, and combinations thereof.

3. The method of claim 1, wherein the at least one FXR agonist and SAMe are administered orally.

4. The method of claim 1, wherein the at least one FXR agonist is selected from the group consisting of obeticholic acid (OCA), cholic acid, EDP-305, GS-9674, LMB-763, tropifexor, EYP-001, TERN-101, AGN-242266, EP-024297, M-480, MET-409, RDX-023, cafestol, fexaramine, GW4064, and combinations thereof.

5. The method of claim 1, wherein the at least one FXR agonist is selected from the group consisting of obeticholic acid (OCA), cholic acid, tropifexor, cafestol, fexaramine, GW4064, and combinations thereof.

6. The method of claim 1, wherein the at least one FXR agonist is selected from the group consisting of obeticholic acid (OCA), tropifexor, GW4064, and combinations thereof.

7. (canceled)

8. (canceled)

9. (canceled)

10. (canceled)

11. (canceled)

12. (canceled)

13. The method of claim 1, wherein the at least one FXR agonist and SAMe are administered sequentially.

14. The method of claim 1, wherein the at least one FXR agonist and SAMe are administered simultaneously.

15. The method of claim 1, wherein SAMe is administered before and after administration of at least one FXR agonist.

16. The method of claim 1, wherein the at least one FXR agonist and SAMe are administered before, during and/or after the subject develops the liver disease.

17. (canceled)

18. (canceled)

19. The method of claim 1, further comprising administering an existing therapy for liver disease to the subject.

20. The method of claim 19, wherein the existing therapy is selected from the group consisting of treatment with vitamin E, pioglitazone and/or life style changes including diet, exercise and weight loss, ursodeoxycholic acid, phenobarbital, cholestyramine, life style changes including diets rich in medium-chain triglycerides, long-chain triglycerides and/or treatment with oral absorbable, fat-soluble vitamin formulation A, D, E, and K supplementation, abstinence from alcohol, cessation of smoking, weight loss and/or treatment with steroids, Naltrexone, Acamprosate, Disulfiram, Topiramate and/or baclofen, eliminating hepatitis B virus or hepatitis C virus in chronic viral hepatitis, abstaining from alcohol, removing heavy metals such as iron in hemochromatosis or copper in Wilson disease, decompressing bile ducts in biliary obstruction and/or treatment with corticosteroids, penicillamine and/or colchicine, Ursodeoxycholic acid (UDCA), liver transplant, treatment with immunosuppressant drugs including methotrexate and/or colchicine and/or treatment with fenofibrate and/or bezafibrate, coffee, and combinations thereof.

21. The method of claim 1, further comprising treating and/or inhibiting and/or reducing a side-effect in the subject.

22. The method of claim 21, wherein the side-effect is associated with or results from the FXR agonist or treatment with the FXR agonist.

23. The method of claim 21, wherein the side-effect is selected from the group consisting of liver cancer, enhanced liver cancer growth, pruritus, and combinations thereof.

24. A method for assessing the efficacy of the treatment of claim 21, comprising comparing the severity of the side-effect in the subject to the severity of the side-effect in a control subject, wherein a decrease in the severity of the side-effect in the subject relative to the control subject is indicative of the efficacy of the treatment.

25. A method for assessing the efficacy of the treatment of claim 21, comprising comparing the liver disease and the side-effect in the subject to the liver disease and the side-effect in a control subject, wherein a decrease in the liver disease and the side-effect in the subject relative to the control subject is indicative of the efficacy of the treatment.

26. A method for treating, reducing and/or inhibiting a side-effect associated with therapeutic use of at least one FXR agonist in a subject, comprising: administering to the subject, a therapeutically effective amount of SAMe.

27. The method of claim 26, further comprising administering a therapeutically effective amount of at least one FXR agonist.

28. (canceled)

29. A pharmaceutical composition, comprising at least one FXR agonist, and SAMe.

30. (canceled)

31. (canceled)

32. (canceled)

33. (canceled)

34. (canceled)
